(19) United States
(12) Patent Application Publication
(10) Pub. No.: US 2003/0073629 A1
(43) Pub. Date: Apr. 17, 2003

(54) Omi and domains thereof that disrupt IAP-caspase interaction

(76) Inventor: Emad S. Alnemri, Ambler, PA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/197,634

(22) Filed: Jul. 15, 2002

Related U.S. Application Data
(60) Provisional application No. 60/340,163, filed on Dec. 14, 2001.
(60) Provisional application No. 60/305,378, filed on Jul. 13, 2001.

(51) Int. Cl. A61K 38/17; C07H 21/04; C12N 9/64; C12P 21/02; C12N 5/06

(52) U.S. Cl. 514/12; 514/44; 536/23.2; 435/69.1; 435/320.1; 435/325; 435/226

(57) ABSTRACT

An isolated nucleic acid molecule comprising a polynucleotide having a sequence encoding a peptide or polypeptide of Omi having at least an N-terminus amino acid sequence of Ala-Val-Pro-Ser and up to 321 contiguous amino acid residues that can be derived from residues 138-458 of SEQ ID NO:1 or a functional variant of each, each of which is capable of specifically binding to at least a portion of an Inhibitor of Apoptosis protein. This peptide can be used in a method to modulate apoptosis or to identify modulators of apoptosis as well as in therapeutic uses.

### STATEMENT OF GOVERNMENT INTEREST

This invention was made in part with funds provided by the United States Government under National Institutes of Health Research Grants AG13487 and CA78890. Accordingly, the United States Government may have certain rights to this invention.

### BACKGROUND OF THE INVENTION

1. Field of the Invention

The present invention relates generally to the modulation of apoptosis, and more particularly, to Omi/Htra2's activation of apoptosis via a caspase-independent manner through its protease activity and a caspase-dependent manner by its ability to disrupt the caspase-Inhibitor of Apoptosis Protein (IAP) interaction, Omi/Htra2 derived and related polypeptides and peptides, and methods of using such polypeptides and peptides to modulate and to identify modulators of apoptosis as well as in therapeutic uses.

2. Description of the Related Art

Apoptosis is a highly conserved cell suicide program essential for development and tissue homeostasis of all metazoan organisms. Changes to the apoptotic pathway that prevent or delay normal cell turnover can be just as important in the pathogenesis of diseases as are abnormalities in the regulation of the cell cycle. Like cell division, which is controlled through complex interactions between cell cycle regulatory proteins, apoptosis is similarly regulated under normal circumstances by the interaction of gene products that either prevent or induce cell death.

Since apoptosis functions in maintaining tissue homeostasis in a range of physiological processes such as embryonic development, immune cell regulation and normal cellular turnover, the dysfunction or loss of regulated apoptosis can lead to a variety of pathological disease states. For example, the loss of apoptosis can lead to the pathological accumulation of self-reactive lymphocytes that occurs with many autoimmune diseases. Inappropriate loss or inhibition of apoptosis can also lead to the accumulation of virally infected cells and of hyperproliferative cells such as neoplastic or tumor cells. Similarly, the inappropriate activation of apoptosis can also contribute to a variety of pathological disease states including, for example, acquired immunodeficiency syndrome (AIDS), neurodegenerative diseases and ischemic injury. Treatments that are specifically designed to modulate the apoptotic pathways in these and other pathological conditions can alter the natural progression of many of these diseases.

Although apoptosis is mediated by diverse signals and complex interactions of cellular gene products, the results of these interactions ultimately feed into a cell death pathway that is evolutionarily conserved between humans and invertebrates. The pathway, itself, is a cascade of proteolytic events analogous to that of the blood coagulation cascade.

Several gene families and products that modulate the apoptotic process have now been identified. Key to the apoptotic program is a family of cysteine proteases termed caspases. The human caspase family includes Ced-3, human ICE (interleukin-1-β converting enzyme) (caspase-1), ICH-1 (caspase-2), CPP32 (caspase-3), ICEᵣₑₗII (caspase-4), ICEᵣₑₗII (caspase-5), Mch2 (caspase-6), ICE-LAP3 (caspase-7), Mch5 (caspase-8), ICE-LAP6 (caspase-9), Mch4 (caspase-10), caspase 11-14, and others.

The caspase proteins share several common features. They are cysteine proteases (named for a cysteine residue in the active site) that cleave their substrates after specific aspartic acid residues (Asp-X). Furthermore, caspases are primarily produced as inactive zymogens, known as procaspases, which require proteolytic cleavage at specific internal aspartate residues for activation. The primary gene product is arranged such that the N-terminal peptide (prodomain) precedes a large subunit domain, which precedes a small subunit domain. The large subunit contains the conserved active site pentapeptide QACXG (X=R, Q, G) (SEQ ID NO: 17), which contains the nucleophilic cysteine residue. The small subunit contains residues that bind the Asp carboxylate side chain and others that determine substrate specificity. Cleavage of a caspase yields the two subunits, the large (generally approximately 20 kD) and the small (generally approximately 10 kD) subunit that associate non-covalently to form a heterodimer, and, in some caspases, an N-terminal peptide of varying length. The heterodimer may combine non-covalently to form a tetramer.

Caspase zymogens are themselves substrates for caspases. Inspection of the interdomain linkages in each zymogen reveals target sites (i.e. protease sites) that indicate a hierarchical relationship of caspase activation. By analyzing such pathways, it has been demonstrated that caspases are required for apoptosis to occur. Moreover, caspases appear to be necessary for the accurate and limited proteolytic events that are the hallmark of classic apoptosis (see Salvesen and Dixit, Cell 91:443-446, 1997). During apoptosis, the initiator caspase zymogens are activated by autocatalytic cleavage, which then activate the effector caspases by cleaving their inactive zymogens (Salvesen and Dixit, Proc. Natl. Acad. Sci. USA 96:10964-10967, 1999; Srinivasula et al., Mol. Cell. 1:949-957, 1998). This characteristic indicates that caspases implicated in apoptosis may execute the apoptotic program through a cascade of sequential activation of initiators and effector procaspases (Salvesen and Dixit, Cell 91:443-446,1997). The initiators are responsible for processing and activation of the effectors. The effectors are responsible for proteolytic cleavage of a number of cellular proteins leading to the characteristic morphological changes and DNA fragmentation that are often associated with apoptosis (reviewed in Cohen, Biochem. J. 326:1-16, 1997; Henkart, Immunity 4:195-201, 1996; Martin and Green, Cell 82:349-352, 1995; Nicholson and Thornberry, TIBS 257:299-306, 1997; Porter et al., BioEssays 19:501-507, 1997; Salvesen and Dixit, Cell 91:443-446, 1997). The first evidence for an apoptotic caspase cascade was obtained from studies on death receptor signaling (reviewed in Fraser and Evan, Cell 85:781-784,1996; Nagata, Cell 88:355-365, 1997) which indicated that the death signal is transmitted in part by sequential activation of the initiator procaspase-8 and the effector procaspase-3 (Boldin et al., Cell 85:803-815, 1996; Fernandes-Alnemri et al., Proc. Natl. Acad. Sci. USA 93:7464-7469, 1996; Muzio et al., Cell 85:817-827, 1996; Srinivasula et al., Proc. Natl. Acad. Sci. USA 93:13706-13711, 1996). More direct evidence was provided when it was demonstrated that the cytochrome c death signal is transmitted through activation of a cascade involving procaspase-9 and caspase-3 (Li et al., Cell 91:479-489, 1997).

The initiator caspase zymogens are activated by adaptor proteins such as FADD and Apaf-1, which associate in a stimulus-dependent manner with the prodomains of these zymogens and promote their activation via oligomerization (Salvesen and Dixit, Proc. Natl. Acad. Sci. USA 96:10964-10967, 1999; Srinivasula et al., Mol. Cell. 1:949-957, 1998). For example, ligands binding to the cell surface death receptors triggers binding of procaspase-8 to FADD and its subsequent activation and release from the death receptor complex. Likewise, release of cytochrome c from the mitochondria in response to apoptotic stimuli such as serum starvation, ionization radiation, DNA damaging agents etc. triggers oligomerization of Apaf-1 in an ATP or dATP dependent manner. The oligomeric Apaf-1 apoptosome then recruits and activates procaspase-9.

Given the potentially irreversible caspase cascade triggered by activation of the upstream initiator caspases, it is crucial that activation of caspases in the cell be tightly regulated. A number of cellular proteins have been shown to modulate caspase activation and activity. One of these, FLAME/FLIP, inhibits death receptor-mediated activation of caspase-8 by binding to FADD (Irmler et al., Nature 388:190-195, 1997; Srinivasula et al., J. Biol. Chem. 272:18542-18545, 1997). Others, such as the anti-apoptotic members of the Bcl-2 family, inhibit Apaf-1-mediated activation of caspase-9 by blocking cytochrome c release from the mitochondria (reviewed in Adams and Cory, Science 281:1322-1326,1998; Green and Reed, Science 281:1309-1312, 1998). Heat shock proteins, Hsp70 and Hsp90, also interfere with the mitochondrial apoptotic pathway by modulating the formation of a functional Apaf-1 apoptosome (Saleh, et al., Nature Cell. Biol. 2:476-483, 2000: Pandey, et al., EMBO J. 19:4310-4322, 2000). Finally, members of the Inhibitor of Apoptosis Protein (IAP) family, such as XIAP, c-IAP-1, and c-IAP-2, block both the death receptor and mitochondrial pathways by inhibiting the activity of the effector caspase-3 and caspase-7 and the initiator caspase-9 (reviewed in Deveraux and Reed, Genes Dev. 13:239-252, 1999).

The IAP proteins were first identified from baculoviruses as proteins that function to suppress host cell death upon viral infection. IAPs have been found in mammals, insects, nematodes and yeast. All IAPs share one or more signature motifs, referred to as BIRs, that are essential for the anti-apoptotic activity associated with these proteins. The BIR motifs have been shown to bind directly to caspases and inhibit their activity (Deveraux and Reed, Genes Dev. 13:239-252, 1999; Deveraux, Q. L. et al. Nature 388:300-304,1997). In the insect Drosophila melanogaster, three proteins known as Reaper, Hid, and Grim have been identified as direct IAP-binding proteins that promote caspase activation by binding to the BIR domain of IAPs and disrupting IAP-caspase interaction (Abrams, J. M. et al., Trends Cell Biol 9:435-440,1999; Vucic, D. et al., Mol Cell Biol 18:3300-3309, 1998; Goyal, L. et al., Embo J 19:589-597, 2000).

Smac/DIABLO, a mitochondrial protein, which is released together with cytochrome c from the mitochondria in response to apoptotic stimuli, was found to promote caspase activation by binding and neutralizing the IAPs in mammals (Du et al., Cell 102:33-42, 2000; Verhagen et al., Cell 102:43-53, 2000). Although Smac does not share sequence homology with Reaper, Hid, and Grim, except for the first four N-terminal residues, which constitute its IAP-binding motif (Wu, G. et al., Nature 408:1008-1012, 2000; Liu, Z. et al., Nature 408:1004-1008, 2000; Srinivasula, S. M. et al., Nature 410:112-116, 2001), it is the only known mammalian functional homolog of these proteins with a similar mode of action. Smac promotes caspase activation and apoptosis by binding to the BIR3 and BIR2 domains of XIAP and disrupting its interaction with caspase-9 and the effector caspases 3 and 7 (Wu, G. et al., Nature 408:1008-1012, 2000; Srinivasula, S. M. et al., Nature 410:112-116, 2001; Srinivasula, S. M. et al., J Biol Chem 2000). Since the mechanism of IAP inhibition of caspases is conserved in mammals and insects, it is thought that other mammalian IAP-binding proteins are still undiscovered.

While the importance of caspases in cell death is well established, a large body of evidence suggests that other types of proteases might also be important (Solary, E. et al., Cell Biol Toxicol 14:121-132, 1998; Johnson, D., Leukemia 14:1695-1703, 2000). For example, inhibitors of serine proteases such as DFP, TLCK, and TPCK can delay or fully inhibit distinct steps of the cell death pathway (Solary, E. et al., Cell Biol Toxicol 14:121-132, 1998; Higuchi, M. et al., Blood 86:2248-2256, 1995). Moreover, inhibition of caspases generally delays but does not completely block cell death by some cell death stimuli.

### SUMMARY OF THE INVENTION

The present invention generally provides nucleic acid molecules that encode peptides or polypeptides of Omi/Htra2 and functional variants of each, peptides or polypeptides of Omi and functional variants of each, and methods of using such peptides to modulate and to identify modulators of apoptosis. However, the present invention does not include precursor Omi or nucleic acid molecules encoding same within the scope of the invention. The invention also provides antibodies that specifically bind an Omi peptide or polypeptide. In addition, the invention provides compositions of Omi peptides or polypeptides, modulators of Omi-mediated apoptosis, or Omi antibodies, and methods of producing such compositions.

In a first aspect of the invention, the present invention provides an isolated nucleic acid molecule comprising a polynucleotide having a sequence encoding a peptide or polypeptide of Omi having an amino acid sequence of Ala-Val-Pro-Ser (SEQ ID NO:3) and up to 321 contiguous amino acid residues that can be derived from residues 138-458 of Omi (SEQ ID NO:1), or a functional variant of each, that specifically binds to a portion of an Inhibitor of Apoptosis Protein (IAP). In certain embodiments, the portion of the IAP bound comprises at least one of the BIR domains of IAP, e.g. BIR1, BIR2, and BIR3, In another embodiment, the peptide or polypeptide binds to a full length IAP.

In another aspect of the invention, the present invention provides an expression vector comprising a nucleic acid molecule of the present invention operatively linked to regulatory elements. Preferably, the regulatory elements include an inducible or a constitutive promoter.

In another aspect of the invention, the present invention provides a host cell containing an expression vector of the present invention.

In a further aspect of the invention, the present invention provides an isolated Omi peptide or polypeptide comprising an amino acid sequence of Ala-Val-Pro-Ser (SEQ ID NO:3) and up to 321 contiguous amino acid residues that can be derived from residues 138-458 of SEQ ID NO:1, or a functional variant of each, that specifically binds to a portion of an Inhibitor of Apoptosis protein. In certain embodiments, the portion of the IAP bound comprises at least one of the BIR domains of IAP, e.g. BIR1, BIR2, and BIR3, In another embodiment, the peptide or polypeptide binds to a full length IAP.

In another aspect of the invention, the present invention provides an isolated nucleic acid molecule comprising a polynucleotide having a sequence encoding a peptide or polypeptide of Omi comprising an amino acid sequence of Gly-Asn-Ser-Gly-Gly-Pro-Leu (SEQ ID NO:11) and up to 314 additional contiguous amino acid residues derived from residues 138-458 of SEQ ID NO:1, wherein said polypeptide induces caspase-independent apoptosis.

In yet another aspect of the invention, the present invention provides an isolated nucleic acid molecule comprising a polynucleotide having a sequence encoding a polypeptide of Omi comprising an amino acid sequence of Met-Ala-Val-Pro-Ser (SEQ ID NO:12), an amino acid sequence of Gly-Asn-Ser-Gly-Gly-Pro-Leu (SEQ ID NO:11), and up to 321 contiguous amino acid residues including SEQ ID NO:11 derived from residues 138-458 of SEQ ID NO:1, wherein said polypeptide induces caspase-independent apoptosis.

In a further aspect of the present invention, the present invention provides an isolated nucleic acid molecule comprising a polynucleotide having a sequence encoding a polypeptide of Omi comprising an amino acid sequence of Ala-Val-Pro-Ser (SEQ ID NO:3), an amino acid sequence of Gly-Asn-Xaa-Gly-Gly-Pro-Leu, wherein Xaa is not Ser, (SEQ ID NO:13), and up to 321 contiguous amino acid residues including SEQ ID NO:13 derived from residues 138-458 of SEQ ID NO:1, wherein said polypeptide fails to have serine protease activity. In one embodiment of this aspect of the present invention, Xaa is an Ala residue.

In one aspect of the present invention, the present invention provides an isolated nucleic acid molecule comprising a polynucleotide having a sequence encoding a polypeptide of Omi comprising the amino acid sequence of residues 134-158 of SEQ ID NO:1 except that the serine at position 306 is mutated and the polypeptide fails to have serine protease activity. In one embodiment of this aspect of the present invention, Xaa is an Ala residue.

In another aspect of the invention, the present invention provides an isolated polypeptide of Omi comprising an amino acid sequence of Gly-Asn-Ser-Gly-Gly-Pro-Leu (SEQ ID NO:11) and up to 314 additional contiguous amino acid residues derived from residues 138-458 of SEQ ID NO:1, wherein said polypeptide induces caspase-independent apoptosis.

In yet another aspect of the invention, the present invention provides an isolated polypeptide of Omi comprising an amino acid sequence of Met-Ala-Val-Pro-Ser (SEQ ID NO:12), an amino acid sequence of Gly-Asn-Ser-Gly-Gly-Pro-Leu (SEQ ID NO:11), and up to 321 contiguous amino acid residues including SEQ ID NO:11 derived from residues 138-458 of SEQ ID NO:1, wherein said polypeptide induces caspase-independent apoptosis.

In a further aspect of the present invention, the present invention provides an isolated polypeptide of Omi comprising an amino acid sequence of Ala-Val-Pro-Ser (SEQ ID NO:3), an amino acid sequence of Gly-Asn-Xaa-Gly-Gly-Pro-Leu, wherein Xaa is not Ser, (SEQ ID NO:13), and up to 321 contiguous amino acid residues including SEQ ID NO:13 derived from residues 138-458 of SEQ ID NO:1, wherein said polypeptide fails to have serine protease activity. In one embodiment of this aspect of the present invention, Xaa is an Ala residue.

In one aspect of the present invention, the present invention provides an isolated polypeptide of Omi comprising the amino acid sequence of residues 134-158 of SEQ ID NO:1 except that the serine at position 306 is mutated and the polypeptide fails to have serine protease activity. In one embodiment of this aspect of the present invention, Xaa is an Ala residue.

In yet another aspect of the invention, the present invention provides a method for inducing caspase-dependent apoptosis in a cell, comprising contacting the cell with either: (a) an isolated Omi peptide or polypeptide comprising an amino acid sequence of Ala-Val-Pro-Ser (SEQ ID NO:3) and up to 321 contiguous amino acid residues that can be derived from residues 138-458 of SEQ ID NO:1, or a functional variant of each, that specifically binds to a portion of an Inhibitor of Apoptosis protein; or (b) an isolated nucleic acid molecule comprising a polynucleotide having a sequence encoding such a peptide or polypeptide, under conditions and for a time sufficient to permit the induction of apoptosis in a cell.

Another aspect of the invention provides a method for inducing caspase-independent apoptosis in a cell, comprising contacting the cell with either: (a) an isolated polypeptide of Omi comprising an amino acid sequence of Gly-Asn-Ser-Gly-Gly-Pro-Leu (SEQ ID NO:11) and up to 314 additional contiguous amino acid residues derived from residues 138-458 of SEQ ID NO:1, wherein said polypeptide induces caspase-independent apoptosis; (b) an isolated polypeptide of Omi comprising an amino acid sequence of Met-Ala-Val-Pro-Ser (SEQ ID NO:12), an amino acid sequence of Gly-Asn-Ser-Gly-Gly-Pro-Leu (SEQ ID NO:11), and up to 321 contiguous amino acid residues including SEQ ID NO:11 derived from residues 138-458 of SEQ ID NO:1, wherein said polypeptide induces caspase-independent apoptosis; or (c) an isolated nucleic acid molecule comprising a polynucleotide having a sequence encoding any such peptide or polypeptide, under conditions and for a time sufficient to permit the induction of apoptosis in a cell.

In another aspect of the invention, the present invention provides a method for identifying an inhibitor or enhancer of a caspase-mediated apoptosis. This method comprises (a) contacting a cell containing a vector expressing an Omi peptide or polypeptide according to the present invention capable of binding an IAP with a candidate inhibitor or candidate enhancer; and (b) detecting cell viability. An increase in cell viability indicates the presence of an inhibitor and a decrease in cell viability indicates the presence of an enhancer.

In yet another aspect of the present invention, the invention provides another method for identifying an inhibitor or enhancer of the caspase-mediated apoptosis process. This method comprises: (a) contacting a cell containing a vector expressing an Omi peptide or polypeptide of the present invention with a candidate inhibitor or candidate enhancer; and (b) detecting the presence of large and small caspase subunits, and therefrom determining the level of caspase processing activity. A decrease in the processing of the procaspase indicates the presence of an inhibitor and an increase in processing indicates the presence of an enhancer. Preferably, the caspase detected is caspase-3, caspase-7 and/or caspase-9.

In another aspect of the present invention, the present invention provides yet another method of identifying an inhibitor or enhancer of caspase-mediated apoptosis. The method comprises: (a) contacting a cell containing a vector expressing an Omi peptide or polypeptide of the present invention with a candidate inhibitor or candidate enhancer; and (b) detecting caspase enzymatic activity. A decrease in enzymatic activity indicates the presence of an inhibitor and an increase in enzymatic activity indicates the presence of an enhancer. Preferably, the caspase enzymatic activity detected is caspase-3, caspase-7 and/or caspase-9. In certain embodiments of the present invention, the caspase enzymatic activity is detected by the presence of a substrate cleavage product produced by a caspase cleavage of a substrate. Preferably, the substrate is acetyl DEVD-aminomethyl coumarin.

In another aspect of the invention, the present invention provides a method for identifying an inhibitor or enhancer of caspase-independent apoptosis. This method comprises (a) contacting a cell containing a vector expressing an Omi peptide or polypeptide according to the present invention that is capable of inducing caspase-independent apoptosis with a candidate inhibitor or candidate enhancer; and (b) detecting cell viability. An increase in cell viability indicates the presence of an inhibitor and a decrease in cell viability indicates the presence of an enhancer.

Another aspect of the present invention provides for an antibody that specifically binds to a peptide or polypeptide of the present invention. In certain aspects of the present invention, the antibody is a polyclonal. In other aspects, the antibody is a monoclonal. In a related aspect, the invention provides for an antibody that specifically binds to an epitope located on the N-terminus of Omi. In a preferred aspect, the epitope includes the amino acid residues Ala-Val-Pro-Ser (SEQ ID NO:3). In certain embodiments, the antibody inhibits the binding of Omi to at least a portion of an IAP. Preferably, the portion of the IAP is at least one BIR domain, e.g., BIR1, BIR2 and/or BIR3, or it can be a full-length IAP.

An additional aspect of the present invention provides for a composition comprising a nucleic acid molecule of the present invention, a peptide of the present invention, or an antibody of the present invention; and a physiologically acceptable carrier.

In other aspects of the present invention, the invention provides assays for identifying a compound that inhibits Omi binding to a Omi-binding molecule. In one aspect, the present invention provides a method comprising contacting a candidate compound with an Omi peptide or polypeptide of the invention in the presence of an Omi-binding molecule, and detecting displacement or inhibition of binding of said Omi-binding molecule from said Omi peptide. In one embodiment, the Omi-binding molecule is at least a portion of an IAP. Preferably, the portion of the IPA is at least one BIR domain, e.g., BIR1, BIR2 and/or BIR3, or it can be a full-length IAP.

In another aspect, the present invention provides a method of identifying a compound that inhibits Omi binding to an Omi-binding molecule that utilizes a functional assay to confirm displacement of said Omi-binding molecule from said Omi peptide or polypeptide. In one aspect of the present invention, a candidate compound is contacted with a peptide or polypeptide of the present invention in the presence of an Omi-binding molecule, at least one caspase or procaspase, cytochrome c, and ATP or dATP. In yet another aspect of the present invention, the method comprises contacting a cell containing a vector expressing a peptide or polypeptide of the present invention with a candidate compound and performing a functional assay that confirms displacement of said Omi-binding molecule from said Omi peptide or polypeptide. In certain embodiments, a functional assay is carried out in the presence of an initiator caspase or procaspase and an effector procaspase and detects the presence of large and small caspase subunits, and therefrom determines the level of caspase processing activity. Generally, a decrease in processing confirms displacement. Preferably, the caspase detected is caspase-3, caspase-7, and/or caspase-9. In another embodiment, the functional assay detects the presence of a substrate cleavage product produced by effector caspase cleavage of a substrate. Preferably, the effector caspase is caspase-3 or caspase-7. An example of a substrate that can be used in this functional assay is acetyl DEVD-aminomethyl coumarin (DEVD-AMC).

In yet another aspect of the present invention, the invention provides an isolated nucleic acid molecule comprising a polynucleotide having a sequence encoding a functional variant of a peptide or polypeptide of the invention. In certain embodiments, a variant has at least 50% identity of said peptide or at least 75% identity of said polypeptide up to 75 residues in length. In another embodiment, a variant has at least 85% identity of said polypeptide over 75 residues in length, and said variant specifically binds to a portion of an IAP.

A further aspect of the invention provides an isolated peptide or polypeptide comprising an amino acid sequence corresponding to a functional variant of a peptide or polypeptide of the invention. In certain embodiments, a variant has at least 50% identity of said peptide or at least 75% identity of said polypeptide up to 75 residues in length. In another embodiment, a variant has at least 85% identity of said polypeptide over 75 residues in length, and said variant specifically binds to a portion of an IAP.

Another aspect of the present invention provides methods of producing a pharmaceutical compound for inhibiting or enhancing caspase-dependent apoptosis in a cell. In certain aspects, the method comprises identifying an inhibitor or enhancer of caspase-mediated apoptosis according to a method of the present invention and purifying said inhibitor or enhancer. In a related aspect, the method comprises identifying a compound that inhibits Omi binding to an Omi-binding molecule according to a method of the present invention and purifying said compound.

Another aspect of the invention proves processes of manufacturing compounds of the invention. In one aspect, the invention provides a process for the manufacture of a compound for inhibiting or enhancing caspase-dependent apoptosis in a cell that includes identifying an inhibitor or enhancer of caspase-mediated apoptosis according to a method of the invention, derivitizing the compound, and optionally repeating at least the identification or derivitization step of the process. In yet another aspect, the process involves identifying a compound that inhibits binding of an Omi peptide or polypeptide according to a method of the invention, derivitizing the compound, and optionally repeating the identification and/or derivitization step.

These and other aspects of the present invention will become evident upon reference to the following detailed description and attached drawings. In addition, the various references set forth herein describe in more detail certain procedures or composition (e.g., plasmids, etc.) and are, therefore, incorporated by reference.

### BRIEF DESCRIPTION OF THE DRAWINGS

FIG. 1 is a scanned image of an autoradiogram representing SDS-PAGE analysis of the interaction of AVPIA-peptide (SEQ ID NO:2) eluates from human HEK293 cell extracts or mouse heart, kidney, liver, or spleen tissues bound to GST-BIR3 fusion protein with ³⁵S-labeled XIAP (upper panel) or a Smac monoclonal antibody (lower panel).

FIG. 2 is a scanned image of a Coomassie stained gel of GST-BIR3 affinity purified proteins.

FIG. 3 is a scanned image of an autoradiogram representing SDS-PAGE analysis of the interaction of HEK293 cell extracts transfected with an Omi precursor expression construct (Omi lane) or empty vector (vector lane) with ³⁵S-labeled XIAP (left panel), a Smac antibody (middle panel), or an Omi polyclonal antibody (right panel).

FIG. 4 is a scanned image of an autoradiogram representing SDS-PAGE analysis of the interaction of AVPIA-peptide (SEQ ID NO:2) eluates from human HEK293 cell extracts of HEK293 cell extracts depleted with a Smac antibody and bound to GST-BIR3 fusion protein or GST-BIR3-E314S with ³⁵S-labeled XIAP (left panel) or a Smac monoclonal antibody (right panel).

FIG. 5 is a bar diagram representation of the structure of the Omi precursor and the location of the AVPS motif (SEQ ID NO:3).

FIG. 6 is a colinear alignment of the N-terminal sequences of Drosophila Reaper (SEQ ID NO:4), Grim (SEQ ID NO:5), and Hid (SEQ ID NO:6) and human caspase-9-p12 (SEQ ID NO:7), Smac (SEQ ID NO:8), and Omi (SEQ ID NO:9).

FIGS. 7A and 7B are scanned images of Coomassie stained gels representing SDS-PAGE analysis of purified C-terminal His6-tagged recombinant wild type mature Omi (WT), Omi-ΔAVPS, Omi-S306A, and mature Smac (7A) and recombinant Omi and Smac proteins with GST-BIR1, GST-BIR2, GST-BIR3, or GST-BIR3-E314S fusion proteins (7B).

FIG. 8 is a scanned image of an autoradiogram representing SDS-PAGE analysis of interaction of XIAP, cIAP1, and cIAP2 with mature Omi and Smac. Immobilized C-terminal His6-tagged mature Omi or Smac, or GST protein was incubated with in vitro-translated ³⁵S-labeled XIAP (first panel), cIAP1 (second panel), or cIAP2 (third panel). The fourth panel is a scanned image of a Coomassie stained gel of the immobilized proteins.

FIG. 9 is a scanned image of a blot representing western blot analysis using an XIAP monoclonal antibody (upper panel) or Flag antibody (middle panel) of 293T cells transfected with constructs encoding C-terminal Flag-tagged Omi wild type or mutant precursors and immunoprecipitated with Flag antibody. The lower panel is a scanned image of a blot representing total extracts probed with the XIAP antibody. Arrows indicate IgG bands.

FIG. 10 is a scanned image of an autoradiogram representing analysis of SDS-PAGE resolved soluble proteins from nuclear, mitochondrial, microsomal, and cytosolic cell fractions bound to GST-BIR3 fusion protein with ³⁵S-labeled XIAP (first panel) or with Omi and Smac antibodies (second and third panels, respectively). Total subcellular fractions were also immunoblotted with antibodies against Cytochrome c oxidase (fourth panel), PARP (fifth panel), or β-actin (sixth panel).

FIG. 11 is a scanned image of a blot representing western blot analysis of supernatant and mitoplasts (pellet) of purified mitochondria probed with Omi (first panel), Smac (second panel), and cytochrome c oxidase (third panel) antibodies.

FIGS. 12A-12I represent confocal micrographs of MCF-7 cells transfected with C-terminal red fluorescent protein (RFP)-tagged Smac precursor (Smac-RFP, 12A), green fluorescent protein (GFP)-tagged Omi precursor (Omi-GFP, 12B), GFP (12D), C-terminal GFP-tagged mature Omi (Omi134-458-GFP, 12E), or Omi precursor (Omi-GFP, 12F), C-terminal RFP-tagged Omi1-60 (Omi-MTS-RFP, 12G) and pEYFP-Mito marker (12H). The right panels in FIGS. 12C and 12F represent merged micrographs schematic diagram of a GFP-Omi fusion protein and its cleavage by caspase-8 to generate a mature cytosolic Omi.

FIG. 13 is a scanned image of a blot representing cytosolic extracts from human Jurkat and HL-60 cells bound to GST-BIR3 and immunoblotted with Omi (top panels), Smac (second panels), Cytochrome c (third panels), or caspase-3 (lower panels) antibodies.

FIG. 14 is a scanned image of a blot representing cytosolic extracts of Jurkat cells treated with TRAIL (1 μg/ml) for the indicated time periods and analyzed by immunoblotting with Omi (top panel), Smac (middle panel), or cytochrome c (bottom panel) antibodies.

FIG. 15 represents immunofluorescence confocal microscopy of untreated or staurosporine-treated (1 μM, 5 hours) Hela cells (upper panels), or untreated or TRAIL-treated (1 μg/ml) MCF-7 cells (lower panels) stained with Omi-specific antibody.

FIG. 16 is a scanned image of a blot representing supernatants (S) and mitochondrial pellets (P) analyzed by immunoblotting with Omi (upper panel), Smac (second panel), cytochrome c (third panel), and cytochrome c oxidase (lower panel) antibodies.

FIG. 17 is a scanned image of an autoradiogram, representing SDS-PAGE analysis of ³⁵S-labeled procaspase-3 following stimulation of 293T S100 extracts (20μg) mixed with purified XIAP (50 ng) with cytochrome c (0.5 μg) plus dATP (1 mM), in the presence of increasing amounts (10, 50, or 300 ng, respectively) of purified mature WT Omi, Omi-ΔAVPS, Omi-S306A, or mature Smac in 10 μl reaction volume. In the fifth lane, S100 extracts were incubated with. 300 ng of WT Omi without XIAP.

FIG. 18 is a bar graphic representation of the percentages of GFP-positive apoptotic HeLa cells following treatment with the indicated doses of staurosporine for five hours, 24 hours after transfection. The Hela cells were transfected with an empty vector or a construct encoding a C-terminally Flag-tagged Omi precursor together with pEGFP-N1 plasmid (Clontech) at a 4:1 ratio. The AVPS motif in the S306A mutant Omi precursor is mutated to a VVAS sequence (SEQ ID NO:10) to prevent binding to IAPs. The Insert represents the expression of mature Omi wild type and Omi S306A mutant in the transfected cells as determined by immunoblotting with Flag antibody.

FIG. 19 is a bar graphic representation of the percentages of GFP-positive apoptotic MCF-7 (left graph) and Hela (right graph) cells transfected with a full-length antisense Omi cDNA (+) in a pRSC-GFP double expression vector or an empty pRSC-GFP vector (−), and treated with Fas, TRAIL, or staurosporine.

FIG. 20 provides schematic diagrams of the Omi-GFP and GFP-IETD-Omi constructs used to assay the ability of Omi to induce apoptosis or to enhance apoptosis by other stimuli. The Omi fusion proteins that induce or enhance apoptosis are marked (+) and those that do not are marked (−).

FIG. 21 is a bar graphic representation of the percentages of GFP-positive apoptotic MCF-7 cells 36 hours following transfection with M-Omi-S/A-GFP or pEGFP-N1 alone or M-Omi-GFP together with empty vector or expression construct encoding XIAP, XIAP-BIR3, or caspase-9DN. The cells were also transfected with M-Omi-GFP expression construct in the presence of zVAD-FMK (20 μM).

FIG. 22 is a bar graphic representation of the percentages of GFP-positive apoptotic Apaf-1 −/− or caspase-9−/− MEFs 36 hours following transfection with pEGFP-N1, M-Omi-GFP, or M-Omi-S/A-GFP.

FIG. 23 is a scanned image of an immunoblot with Flag-HRP antibody of MCF-7 and 293T cells 24 hours following transfected with pEGFP or M-Omi-Flag, GFP-IETD-AVP (AVP) or GFP-IETD-SSA (SSA) expression constructs in the presence or absence of zVAD-FMK (20μM). M-Omi-Flag is similar to the M-Omi-GFP shown in FIG. 20, except that the GFP tag was replaced with a Flag tag.

FIG. 24 is a scanned image of an immunoblot with Flag-HRP antibody of 293T cells extracts precipitated with GST-BIR3 24 hours after transfection with the indicated expression constructs extracts.

FIG. 25 is a bar graphic representation of the percentages of the determined round apoptotic MCF-7 cells that were left untreated (−) or treated with TRAIL (+) for 16 hours 24 hours following transfection with the indicated expression constructs.

### DETAILED DESCRIPTION OF THE INVENTION

Prior to setting forth the invention, it may be helpful to an understanding thereof to set forth definitions of certain terms that will be used hereinafter.

An “isolated nucleic acid molecule” refers to a polynucleotide molecule in the form of a separate fragment or as a component of a larger nucleic acid construct, which has been separated from its source cell (including the chromosome it normally resides in) at least once, and preferably in a substantially pure form. Nucleic acid molecules may be comprised of a wide variety of nucleotides, including DNA, RNA, nucleotide analogues, or a combination thereof.

A “functional” peptide or polypeptide, as used herein, refers to a peptide or polypeptide comprising at least the N-terminus amino acid sequence of Omi comprising Ala-Val-Pro-Ser (SEQ ID NO:3) and/or that retains at least one biological or functional activity associated with N-terminal domain of Omi. This functional peptide or polypeptide can also include up to 321 contiguous amino acid residues that can be derived from residues 138-458 of SEQ ID NO:1. The N-terminus sequence of Omi (SEQ ID NO:3) may be located at the N-terminus of a functional peptide or polypeptide, or it may be preceded by one or more amino acid residues, such as, for example, a single Met residue. Preferably, the biological or functional activity is the specific binding to at least a portion of an Inhibitor of Apoptosis Protein (IAP). Preferably this portion of the IAP to which the peptide or polypeptide specifically binds is at least a BIR domain. In one embodiment, this BIR domain is BIR3. In other embodiments, this BIR domain is BIR1 or BIR2. In certain embodiments, the peptide or polypeptide specifically binds to a full length IAP. Such functional peptides or polypeptides may comprise, consist essentially of, or consist of an amino acid sequence having at least an N-terminus amino acid sequence of Ala-Val-Pro-Ser (SEQ ID NO:3) or Met-Ala-Val-Pro-Ser (SEQ ID NO:12) and up to 321 contiguous amino acid residues that can be derived from residues 138-458 of SEQ ID NO:1.

As used herein, a “peptide” is an amino acid sequence of between four and ten contiguous amino acids, including all integer values in between, i.e. 4, 5, 6, 7, 8, 9 and 10 contiguous amino acids. A “polypeptide” is an amino acid sequence of more than ten contiguous amino acids up to and including “mature” Omi, including all integer values in between, e.g. 11, 15, 20, 30, 40, 60, 75, 100, 125, 150, 160, 175, 190, 200 or more contiguous amino acids. “Mature” Omi is a Omi polypeptide comprising residues 134-458 of SEQ ID NO:1 and without contiguous amino acid residues 1 to 133 of SEQ ID NO:1. Amino acid residues 1 to 133 include the mitochondrial targeting sequence (MTS). Accordingly, once residues 1 to 133 are removed from the Omi precursor, increases levels of the “mature” Omi are found in the cytoplasm.

References to Omi herein are intended to include peptides or polypeptides of any origin which are substantially homologous to and which are biologically or functionally equivalent to the Omi peptides and polypeptides characterized and described herein.

A “caspase” refers to a cysteine protease that specifically cleaves proteins after Asp residues. Caspases are initially expressed as zymogens, in which a large subunit is N-terminal to a small subunit. Caspases are generally activated by cleavage at internal Asp residues. These proteins have been identified in many eukaryotes, including C. elegans, Drosophila, mouse, and humans. Currently, there are at least 14 known caspase genes, named caspase-1 through caspase-14. Caspases are found in a myriad of organisms, including human, mouse, insect (e.g., Drosophila), and other invertebrates (e.g., C. elegans). In Table 1, ten human caspases are listed along with their alternative names.

TABLE 1

| Caspase | Alternative name |
|---|---|
| Caspase-1 | ICE |
| Caspase-2 | ICH-1 |
| Caspase-3 | CPP32, Yama, apopain |
| Caspase-4 | ICEᵣₑₗll; TX, ICH-2 |
| Caspase-5 | ICEᵣₑₗlll; TY |
| Caspase-6 | Mch2 |
| Caspase-7 | Mch3, ICE-LAP3, CMH-1 |
| Caspase-8 | FLICE; MACH; Mch5 |
| Caspase-9 | ICE-LAP6; Mch6 |
| Caspase- | Mch4, FLICE-2 |

A molecule is said to “specifically bind” to a particular peptide or polypeptide if it binds at a detectable level with the particular peptide polypeptide, but does not bind detectably with another polypeptide containing an unrelated sequence. An “unrelated sequence,” as used herein, refers to a sequence that is at most 10% identical to a reference sequence.

The term “in vitro” refers to cell free systems.

The term “derivitizing” or “derivatizing” refers to standard types of chemical modifications of a compound to produce another structurally related compound typically carried out in the process of compound optimization. The resulting structurally related compound is referred to as a “derivative compound.”

The current invention includes compositions comprising, consisting essentially of, and consisting of nucleic acids encoding and peptides and polypeptides corresponding to a peptide or polypeptide of Omi, or variants thereof. In certain embodiments, said peptides or polypeptides retain at least one functional activity associated with the N-terminal domain of Omi. In one embodiment, the peptide or polypeptide of Omi includes at least an N-terminus amino acid sequence of Ala-Val-Pro-Ser and up to 321 contiguous amino acid residues derived from residues 138-458 of SEQ ID NO:1 that specifically binds to at least a portion of an Inhibitor of Apoptosis Protein (IAP). The current invention also includes compositions comprising, consisting essentially of, and consisting of nucleic acids encoding and polypeptides corresponding to a peptide or polypeptide of Omi including residues corresponding to the serine protease domain or active site located at amino acids 304-310 of SEQ ID NO:1, or variants or mutants thereof, including mutants lacking serine protease activity. In addition, the invention identifies methods of using the peptides of the invention for apoptosis modulation and to identify modulators of caspase-mediated or caspase-independent apoptosis, as well as in therapeutic uses.

A. Omi Nucleic Acid Molecules

The present invention provides nucleic acid molecules that encode peptides of Omi or variants thereof that retain at least one functional activity associated with an N-terminal domain of Omi. In one embodiment, the peptide or polypeptide of Omi comprises at least an N-terminus amino acid sequence of Ala-Val-Pro-Ser (SEQ ID NO:3) and up to 321 contiguous amino acid residues derived from residues 138-458 of SEQ ID NO:1. The invention includes any and all nucleic acid sequences that encode this Omi peptide or variants thereof. In addition, the invention includes specific mutants of this Omi peptide or polypeptide. The Omi peptide or polypeptide may include the serine protease active site region located at residues 304-310 of SEQ ID NO:1. Alternatively, the Omi peptide or polypeptide may lack this region or contain a mutation within residues 304-310 that abolishes serine protease activity. In particular, an Omi polypeptide of the invention may contain residues corresponding to amino acids 304-310 of SEQ ID NO:1, wherein the Ser at residue 306 is replaced with a different amino acid residue, such as Ala, for example.

Omi peptides may be identified as such by any means known in the art, including sequence and functional analysis. Preferably, the Omi peptide or variant has the ability to bind an Inhibitor of Apoptosis protein (IAP) or a portion of an IAP. In certain embodiments the portion of the IAP is a BIR1, a BIR2, and/or a BIR3 domain. The ability of an Omi peptide or polypeptide, or variant thereof, to bind to a portion of an IAP may be determined by a variety of protein binding assays known in the art, including in vitro binding assays using Glutathion-sepharose fusion proteins or co-immunoprecipitation of in vitro translated polypeptides using antibodies specific to one polypeptide, for example. In addition, in vivo binding may be assayed by co-immunoprecipitation of extracts from cells expressing both polypeptides. The specificity of binding may be determined by comparing binding of an experimental sample to binding of an appropriate control sample. Suitable controls include, for example, polypeptides containing mutated binding sites and unrelated proteins. Detailed methods are provided in Examples 1 and 2.

The nucleic acid sequence of full-length Omi and the encoded protein sequence are available in GenBank/EBI DataBank at Accession No. XM_{—}035219. The nucleotide sequence encoding the precursor Omi has been incorporated into the application in SEQ ID NO:14, and the encoded protein has been incorporated into the application in SEQ ID NO:1.

Omi nucleic acid molecules may be isolated from genomic DNA or cDNA according to practices known in the art (see Sambrook et al., Molecular Cloning: A Laboratory Manual, Cold Spring Harbor Press, 1989).

Other methods may also be utilized to obtain Omi nucleic acid molecules. One preferred method is to perform polymerase chain reaction (PCR) to amplify a Omi nucleic acid molecule from cDNA or genomic DNA using oligonucleotide primers corresponding to the 5′ and 3′ ends of Omi nucleic acid molecules or regions thereof. Detailed methods of PCR cloning may be found in Ausubel, et al., Current Protocols in Molecular Biology, Greene Publishing Associates and Wiley-Interscience, NY, 1995, for example.

Polynucleotides of the invention may also be made using the techniques of synthetic chemistry given the sequences disclosed herein. The degeneracy of the genetic code permits alternate nucleotide sequences that will encode the same amino acid sequences. All such nucleotide sequences are within the scope of the present invention.

Nucleic acid sequences encoding Omi peptides may be fused to sequences encoding a secretion signal or sequences encoding the MTS sequence can be removed, whereby the resulting polypeptide is a precursor protein that is subsequently processed and secreted. The resulting processed Omi polypeptide may be recovered from the cell lysate, periplasmic space, phloem, or from the growth or fermentation medium. Secretion signals suitable for use are widely available are well known in the art (e.g., von Heijne, J. Mol. Biol. 184:99-105, 1985).

The Omi nucleic acid molecules of the subject invention also include variants (including alleles) of the native nucleic acid molecules of the present invention. Variants of the Omi nucleic acid molecules provided herein include natural variants (e.g., polymorphisms, splice variants or mutants) and those produced by genetic engineering (e.g., substitutions, deletions or addition of residues). Many methods for generating mutants have been developed (see generally, Ausubel et al., supra). Preferred methods include alanine scanning mutagenesis and PCR generation of mutants using an oligonucleotide containing the desired mutation to amplify mutant nucleic acid molecules. Variants generally have at least 70% or 75% nucleotide identity to the native sequence, preferably at least 80%-85%, and most preferably at least 90%, 92%, 93%, 94%, 95%, 96%, 97%, 98% or 99% nucleotide identity. The identity algorithms and settings that may be used are defined herein infra, but may also include using computer programs which employ the Smith-Waterman algorithm, such as the MPSRCH program (Oxford Molecular), using an affine gap search with the following parameters: a gap open penalty of 12 and a gap extension penalty of 1. A preferred method of sequence alignment uses the GCG PileUp program (Genetics Computer Group, Madison, Wis.) (Gapweight: 4, Gaplength weight: 1). In certain embodiments, the alignment algorithm utilizes default parameters. Further, a nucleotide variant will typically be sufficiently similar in sequence to hybridize to the reference sequence under moderate or stringent hybridization conditions. For nucleic acid molecules over about 500 bp, stringent conditions include a solution comprising about 1 M Na⁺ at 25° to 30° C. below the Tm; e.g., 5×SSPE, 0.5% SDS, at 65° C.; see Ausubel et al., Current Protocols in Molecular Biology, Greene Publishing, 1995; Sambrook et al., Molecular Cloning: A Laboratory Manual, Cold Spring Harbor Press, 1989). Typically, homologous polynucleotide sequences can be confirmed by hybridization under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each, homologous sequences can be identified which contain at most about 25-30% basepair mismatches. More preferably, homologous nucleic acid strands contain 15-25% basepair mismatches, even more preferably 5-15% basepair mismatches.

Typically, for stringent hybridization conditions, a combination of temperature and salt concentration should be chosen that is approximately 12-20° C. below the calculated Tm of the hybrid under study. The Tm of a hybrid between a nucleotide sequence of the present invention and a polynucleotide sequence which is 70%, 75%, 80%, 85%, 90%, 95%, 96%, 97%, 98%, or 99% identical can be calculated, for example, using the equation of Bolton and McCarthy, Proc. Natl. Acad. Sci. U.S.A. 48, 1390 (1962):

Tₘ=81.5° C.−16.6(log₁₀[Na⁺])+0.41(% G+C)−0.63(% formamide)−600/l),

where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C. Suitable moderately stringent conditions include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5×and 0.2×SSC containing 0.1% SDS.

Nucleic acid sequences which are substantially the same as the nucleic acid sequences encoding Omi are included within the scope of the invention. Such substantially same sequences may, for example, be substituted with codons optimized for expression in a given host cell such as E. coli. The present invention also includes nucleic acid sequences that will hybridize to sequences that encode viral, human, or murine Omi or complements thereof. The invention includes nucleic acid sequences encoding peptides and polypeptides of at least the N-terminus of the Omi protein. Deletions, insertions and/or nucleotide substitutions within a Omi nucleic acid molecule are also within the scope of the current invention. Such alterations may be introduced by standard methods known in the art such as those described in Ausubel et al., supra. Also included are nucleic acid sequences encoding functional equivalents of an Omi peptide or polypeptide. In addition, the invention includes nucleic acids that encode polypeptides that are recognized by antibodies that bind an Omi peptide, polypeptide, functional variants of each, or functional equivalents of each.

Polynucleotide molecules of the invention can comprise at least 12, 15, 18, 21, 24, 27, 30, 33, 36, 39, 42, 54, 60, 66, 72, 84, 90, 100, 120, 140, 200, 240, 250, 300, 330, 400, 420, 500, 535, 580, 600, 655, 700, 760, 800, 850, 900, 970 or more contiguous nucleotides derived from nucleotide position 400 up to and including nucleotide position 1374 of SEQ ID NO:14 or the complements thereof.

Polynucleotide molecules of the invention also include molecules that encode single-chain antibodies which specifically bind to the disclosed peptides or polypeptides or specifically bind to mRNA encoding the disclosed peptides or polypeptides or fusion proteins comprising amino acid sequences of the disclosed proteins.

B. Omi Peptides

The present invention includes one embodiment wherein the polypeptide or peptide sequences are derived from at least the N-terminus of Omi, but the embodiment does not include the full-length precursor Omi. In certain embodiments, the peptides comprise, consist essentially of or consist of the N-terminus amino acid sequence of Ala-Val-Pro-Ser (SEQ ID NO:3) and up to 321 contiguous amino acids that are derived from residues 138 to 458 of SEQ ID NO:1 and that specifically bind to a portion of an Inhibitor of Apoptosis Protein (IAP) (e.g., XIAP and CIAP) or to a full length IAP. In other embodiments, such functional peptides or polypeptides comprise, consist essentially of, or consist of the N-terminus amino acid sequence of Ala-Val-Pro-Ser and up to 321 contiguous amino acids that are derived from residues 138 to 458 of SEQ ID NO:1 including all integer values in between (e.g. 4, 5, 7, 9, 10, 15, 20, 30, 40, 50, 60, 70, 80, 90, 100, 110, 120, 125, 130, 140, 150, 155, 160, 170, 180, 190, 200, 210, 220, 230, 250, 275, 300, 320 or more contiguous amino acids) and have at least about 75% or 80% amino acid sequence identity with a peptide derived from residues 134-458 of SEQ ID NO:1. In other embodiments, such a functional peptide or polypeptide comprises the N-terminus amino acid sequence of Ala-Val-Pro-Ser and up to 321 contiguous amino acids that are derived from residues 138 to 458 of SEQ ID NO:1, including all integer values in between, and have at least about 75%, 80%, 85%, 90%, 92%, 95%, 97%, 98% or 99% amino acid sequence identity with a peptide or polypeptide derived from at least residues 134-458 of SEQ ID NO:1. In certain embodiments, the amino acid Met may be inserted immediately amino-terminal of the polypeptide or peptide sequence derived from the amino-terminus of Omi. The aforementioned identities may be calculated with any one of the algorithms herein described.

In another embodiment, a peptide or polypeptide of the present invention comprises, consists essentially of, or consists of an isolated peptide or polypeptide of Omi having at least an amino acid sequence of Gly-Asn-Ser-Gly-Gly-Pro-Leu (SEQ ID NO:11) and up to 314 contiguous amino acid residues that can be derived from residues 138-458 of SEQ ID NO:1, or a functional variant of each region, each of which is capable of inducing apoptosis. The sequence of Gly-Asn-Ser-Gly-Gly-Pro-Leu is the serine protease active site. Alternatively, a peptide or polypeptide of the present invention may comprise, consist essentially of, or consist of an isolated peptide or polypeptide of Omi having a mutated serine protease active site, such as the amino acid sequence of Gly-Asn-Xaa-Gly-Gly-Pro-Leu, wherein Xaa is any amino acid residue except Ser (SEQ ID NO:13). Such a mutated peptide or polypeptide may comprise up to 314 contiguous amino acid residues derived from residues 138-458 of SEQ ID NO:1, or a functional variant, such that the mutant lacks or possesses diminished serine protease activity, yet is still capable of inducing apoptosis via a caspase-dependent mechanism. The aforementioned mutants may be naturally-occurring or engineered, preferably by substituting or deleting an amino acid within the serine protease active site region located at residues 304-310 of SEQ ID NO:1. A preferred method of destroying serine protease activity is by replacing the Ser at residue 306 of SEQ ID NO:1 with another amino acid, such as Ala, for example.

The current invention encompasses all variants (including alleles) of the native Omi peptide or polypeptide sequences as defined in the present invention that retains at least one biological or functional activity associated with N-terminal domain of Omi. Preferably the biological or functional activity is the specific binding to at least a portion of an Inhibitor of Apoptosis Protein (IAP). Such functional variants may result from natural polymorphisms or may be synthesized by recombinant methodology, and differ from wild-type peptides by one or more amino acid substitutions, insertions, deletions, or the like. Amino acid changes in functional variants of Omi peptides or polypeptides may be conservative substitutions. Guidance in determining which amino acid residues can be substituted, inserted, or deleted without abolishing biological or immunological activity can be found using computer programs well known in the art, such as DNASTAR software. Preferably, amino acid changes in secreted functional variants are conservative amino acid changes, i.e., substitutions of similarly charged or uncharged amino acids. It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting variant. Whether an amino acid change results in a functional secreted protein or polypeptide can readily be determined by testing the altered protein or polypeptide in a functional assay, for example, as disclosed in U.S. Pat. No. 5,654,173 and described in detail below.

A conservative amino acid change involves substitution of one amino acid for another amino acid of a family of amino acids with structurally related side chains. Naturally occurring amino acids are generally divided into four families: acidic (aspartate, glutamate), basic (lysine, arginine, histidine), non-polar (alanine, valine, leucine, isoleucine, proline, phenylaianine, methionine, tryptophan), and uncharged polar (glycine, asparagine, glutamine, cysteine, serine, threonine, tyrosine) amino acids. Phenylalanine, tryptophan, and tyrosine are sometimes classified as aromatic amino acids. Non-naturally occurring amino acids can also be used to form protein variants of the invention.

In the region of homology to the native sequence, functional variants should preferably have at least 70-99% amino acid identity, including all integer values in between, e.g., at least 70%, 75%, 80%, 90%, 92%, 95%, 97%, 98% or 99% amino acid identity. In certain embodiments, the peptide or polypeptide sequence is compared to a test sequence, or, when necessary, a particular domain is compared to a test sequence to determine percent identity, typically by utilizing default parameters. Such amino acid sequence identity may be determined by standard methodologies, including those set forth supra as well as the use of the National Center for Biotechnology Information BLAST 2.0 search methodology (Altschul et al., J. Mol. Biol. 215:403-10, 1990). In one embodiment BLAST 2.0 is utilized with default parameters. A preferred method of sequence alignment uses the GCG PileUp program (Genetics Computer Group, Madison, Wis.) (Gapweight: 4, Gaplength weight: 1). The pileUp program creates a multiple sequence alignment from a group of related sequences using progressive, pairwise alignments. PileUp creates a multiple sequence alignment using the progressive alignment method of Feng and Doolittle (J. Mol. Evol. 25:351-360, 1987) and is similar to the method described by Higgins and Sharp (CABIOS 5:151-153,1989). Further, whether an amino acid change results in a functional peptide can be readily determined by assaying biological properties of the disclosed peptides. For example, the biological properties of Omi functional variants can be assayed by determining whether they bind to at least a portion of a IAP, as described in Example 2, or by examining their effects of apoptosis and/or caspase activation, as described in Examples 4 and 5.

Omi functional peptides or polypeptides can include hybrid and modified forms of Omi peptides or polypeptides such as, but not limited to, fusion polypeptides. Omi fusion polypeptides include peptides or polypeptides of Omi fused to amino acid sequences comprising one or more heterologous polypeptides. Such heterologous polypeptides may correspond to naturally occurring polypeptides of any source or may be recombinantly engineered amino acid sequences. Fusion proteins are useful for purification, generating antibodies against amino acid sequences, and for use in various assay systems. For example, fusion proteins can be used to identify proteins or a domain of that protein which interacts with a peptide or polypeptide of the invention or which interferes with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of one or more of the disclosed proteins can be used to target other protein domains to cellular locations in which the domains are not normally found, such as bound to a cellular membrane or secreted extracellularly.

A fusion protein comprises two protein segments fused together by means of a peptide bond. Amino acid sequences for use in fusion proteins of the invention can be selected from any contiguous amino acid sequences as herein described.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex A DNA binding domain (DBD) fusions, GAL4 DNA binding domain fusions, and herpes simplex virus (HSV) BP16 protein fusions.

These fusions can be made, for example, by covalently linking two protein segments or by standard procedures in the art of molecular biology. Recombinant DNA methods can be used to prepare fusion proteins, for example, by making a DNA construct which comprises coding sequences derived from SEQ ID NO:1 in proper reading frame with nucleotides encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

These heterologous polypeptides may be of any length and may include one or more amino acids. In certain embodiments, Omi fusion proteins may be produced to facilitate expression or purification. For example, a Omi polypeptide may be fused to maltose binding protein or glutathione-S-transferase. In other embodiments, Omi fusion proteins may contain an epitope tag to facilitate identification or purification. One example of a tag is the FLAG epitope tag (Kodak). Omi variants may have certain amino acids which have been deleted, replaced or modified. Variants can also include post-translational modifications.

C. Vectors, Host Cells, and Means of Expressing and Producing Protein

The present invention encompasses vectors comprising regulatory elements linked to Omi nucleic acid sequences. Such vectors may be used, for example, in the propagation and maintenance of Omi nucleic acid molecules or the expression and production of Omi peptides or polypeptides or functional variants of each or functional equivalents of each and nucleic acid molecules. Vectors may include, but are not limited to, plasmids, episomes, baculovirus, retrovirus, lentivirus, adenovirus, and parvovirus including adeno-associated virus.

Omi may be expressed in a variety of host organisms. In certain embodiments, Omi is produced in mammalian cells, such as CHO, COS-7, or 293 cells. Other suitable host organisms include bacterial species (e.g., E. coli and Bacillus), other eukaryotes such as yeast (e.g., Saccharomyces cerevisiae), plant cells and insect cells (e.g., Sf9). Vectors for these hosts are well known in the art.

A DNA sequence encoding Omi, or an Omi peptide or polypeptide, is introduced into an expression vector appropriate for the host. The sequence is derived from an existing clone or synthesized. As described herein, a fragment of the coding region may be used. A preferred means of synthesis is amplification of the nucleic acid molecule encoding the peptide of the present invention from cDNA, genomic DNA, or a recombinant clone using a set of primers that flank the desired portion of the protein. Restriction sites are typically incorporated into the primer sequences and are chosen with regard to the cloning site of the vector. If necessary, translational initiation and termination codons can be engineered into the primer sequences. The sequence of Omi can be codon-optimized for expression in a particular host. For example, a Omi isolated from a human cell that is expressed in a fungal host, such as yeast, can be altered in nucleotide sequence to use codons preferred in yeast. Further, it may be beneficial to insert a traditional AUG initiation codon at the CUG initiation positions to maximize expression, or to place an optimized translation initiation site upstream of the CUG initiation codon. Accordingly, such codon-optimization may be accomplished by methods such as splice overlap extension, site-directed mutagenesis, automated synthesis, and the like.

At minimum, the vector must contain a promoter sequence. As used herein, a “promoter” refers to a nucleotide sequence that contains elements that direct the transcription of a linked gene. At minimum, a promoter contains an RNA polymerase binding site. More typically, in eukaryotes, promoter sequences contain binding sites for other transcriptional factors that control the rate and timing of gene expression. Such sites include TATA box, CMT box, POU box, AP1 binding site, and the like. Promoter regions may also contain enhancer elements. When a promoter is linked to a gene so as to enable transcription of the gene, it is “operatively linked”.

Typical regulatory elements within vectors include a promoter sequence that contains elements that direct transcription of a linked gene and a transcription termination sequence. The promoter may be in the form of a promoter that is naturally associated with the gene of interest. Alternatively, the nucleic acid may be under control of a heterologous promoter not normally associated with the gene. For example, tissue specific promoter/enhancer elements may be used to direct expression of the transferred nucleic acid in repair cells. In certain instances, the promoter elements may drive constitutive or inducible expression of the nucleic acid of interest. Mammalian promoters may be used, as well as viral promoters capable of driving expression in mammalian cells. Examples of other regulatory elements that may be present include secretion signal sequences, origins of replication, selectable markers, recombinase sequences, enhancer elements, nuclear localization sequences (NLS) and matrix association regions (MARS).

The expression vectors used herein include a promoter designed for expression of the proteins in a host cell (e.g., bacterial). Suitable promoters are widely available and are well known in the art. Inducible or constitutive promoters are preferred. Such promoters for expression in bacteria include promoters from the T7 phage and other phages, such as T3, T5, and SP6, and the trp, lpp, and lac operons. Hybrid promoters (see U.S. Pat. No. 4,551,433), such as tac and trc, may also be used. Promoters for expression in eukaryotic cells include the P10 or polyhedron gene promoter of baculovirus/insect cell expression systems (see, e.g., U.S. Pat. Nos. 5,243,041, 5,242,687, 5,266,317, 4,745,051, and 5,169,784), MMTV LTR, CMV IE promoter, RSV LTR, SV40, metallothionein promoter (see, e.g., U.S. Pat. No. 4,870,009) and the like.

The promoter controlling transcription of Omi may itself be controlled by a repressor. In some systems, the promoter can be derepressed by altering the physiological conditions of the cell, for example, by the addition of a molecule that competitively binds the repressor, or by altering the temperature of the growth media. Preferred repressor proteins include, but are not limited to, the E. coli lacl repressor responsive to IPTG induction, the temperature sensitive λcl857 repressor, and the like. The E. coli lad repressor is preferred.

In other preferred embodiments, the vector also includes a transcription terminator sequence. A “transcription terminator region” has either a sequence that provides a signal that terminates transcription by the polymerase that recognizes the selected promoter and/or a signal sequence for polyadenylation.

Preferably, the vector is capable of replication in the host cells. Thus, when the host cell is a bacterium, the vector preferably contains a bacterial origin of replication. Preferred bacterial origins of replication include the f1-ori and col E1 origins of replication, especially the ori derived from pUC plasmids. In yeast, ARS or CEN sequences can be used to assure replication. A well-used system in mammalian cells is SV40 ori.

The plasmids also preferably include at least one selectable marker that is functional in the host. A selectable marker gene includes any gene that confers a phenotype on the host that allows transformed cells to be identified and selectively grown. Suitable selectable marker genes for bacterial hosts include the ampicillin resistance gene (Amp^{r}), tetracycline resistance gene (Tc^{r}) and the kanamycin resistance gene (Kan^{r}). The kanamycin resistance gene is presently preferred. Suitable markers for eukaryotes usually require a complementary deficiency in the host (e.g., thymidine kinase (tk) in tk- hosts). However, drug markers are also available (e.g., G418 resistance and hygromycin resistance).

The sequence of nucleotides encoding Omi may also include a secretion signal or the mitochondrial targeting sequence (MTS) sequence can be removed, whereby the resulting peptide or polypeptide is a precursor protein processed and secreted. The resulting processed peptide or polypeptide may be recovered from the periplasmic space, the growth medium, phloem, etc. Secretion signals suitable for use are widely available and are well known in the art (von Heijne, J. Mol. Biol. 184:99-105, 1985). Prokaryotic and eukaryotic secretion signals that are functional in E. coli (or other host) may be employed. The presently preferred secretion signals include, but are not limited to, those encoded by the following E. coli genes: pelB (Lei et al., J. Bacteriol. 169:4379, 1987), phoA, ompA, ompT, ompF, ompC, beta-lactamase, and alkaline phosphatase.

One skilled in the art appreciates that there are a wide variety of suitable vectors for expression in bacterial cells that are readily obtainable. Vectors such as the pET series (Novagen, Madison, Wis.), the tac and trc series (Pharmacia, Uppsala, Sweden), pTTQ18 (Amersham International plc, England), pACYC 177, the pGEX series, and the like are suitable for expression of Omi. Baculovirus vectors, such as pBlueBac (see, e.g., U.S. Pat. Nos. 5,278,050, 5,244,805, 5,243,041, 5,242,687, 5,266,317, 4,745,051, and 5,169,784; available from Invitrogen, San Diego) may be used for expression in insect cells, such as Spodoptera frugiperda sf9 cells (see U.S. Pat. No. 4,745,051). The choice of a bacterial host for the expression of Omi is dictated in part by the vector. Commercially available vectors are paired with suitable hosts.

A wide variety of suitable vectors for expression in eukaryotic cells are also available. Such vectors include pCMVLacl, pXT1 (Stratagene Cloning Systems, La Jolla, Calif.); pCDNA series, pREP series, pEBVHis (Invitrogen, Carlsbad, Calif.). In certain embodiments, Omi gene is cloned into a gene targeting vector, such as pMC1 neo, a pOG series vector (Stratagene Cloning Systems).

Omi expression vectors may be introduced into host cells by a variety of methods well known in the art, depending upon the type of vector and corresponding host cell. For example, eukaryotic host cells may be transfected with plasmid or episomal vectors or infected with viral vectors. Bacterial or yeast host cells may be transformed with plasmid vectors, for example. Host cells may contain expression vectors transiently, as episomes, or stably integrated into the host cell genome. The Omi peptides or polypeptides may be isolated from cells or host cells containing an Omi expression vector by standard methods, such as affinity chromatography, size exclusion chromatography, metal ion chromatography, ionic exchange chromatography, HPLC, and other known protein isolation methods. (See generally Ausubel et al., supra; Sambrook et al., supra). An isolated purified peptide or polypeptide usually gives a single band on SDS-PAGE when stained with Coomassie blue.

The Omi peptides or polypeptides, as discussed earlier, may be expressed as fusion proteins to aid in purification. Such fusions may be, for example, glutathione-S-transferase fusions, Hex-His fusions, or the like such that the fusion construct may be easily isolated. With regard to Hex-His fusions, such fusions can be isolated by metal-containing chromatography, such as nickel-coupled beads. Briefly, a sequence encoding His₆ is linked to a DNA sequence encoding Omi. Although the His₆ sequence can be positioned anywhere in the molecule, preferably it is linked at the 3′ end immediately preceding the termination codon. The fusion may be constructed by any of a variety of methods. A convenient method is amplification of the Omi gene using a downstream primer that contains the codons for His₆.

The purified Omi peptide or polypeptide may be used in various assays to screen for modulators (i.e., inhibitors or enhancers) of apoptosis. These assays may be performed in vitro or in vivo and utilize any of the methods described herein or that are known in the art. The protein may also be crystallized and subjected to X-ray analysis to determine its 3-dimensional structure. The Omi peptides may also be used as immunogens for raising antibodies.

Recombinant Omi peptides or polypeptides may be produced by expressing the DNA sequences provided in the invention. Using methods known in the art, a Omi peptide or polypeptide expression vector may be constructed, transformed into a suitable host cell, and conditions suitable for expression of an Omi peptide by the host cell established. One skilled in the art will appreciate that there are a wide variety of suitable vectors for expression in bacterial cells (e.g. pET series (Novagen, Madison, Wis.)), insect cells (e.g. pBlueBac (Invitrogen, Carlsbad, Calif.)), and eukaryotic cells (e.g. pCDNA and pEBVHis (Invitrogen, Carlsbad, Calif.)). In certain embodiments, the Omi nucleic acid molecule may be cloned into a gene targeting vector such as pMC1 neo (Stratagene, La Jolla, Calif.). Synthetic chemistry methods, such as solid phase peptide synthesis can also be used to synthesize proteins, fusion proteins, or polypeptides of the invention.

The resulting expressed peptide or polypeptide can be purified from the culture medium or from extracts of the cultured cells. Methods of protein purification such as affinity chromatography, ionic exchange chromatography, HPLC, size exclusion chromatography, ammonium sulfate crystallization, electrofocusing, or preparative gel electrophoresis are well known and widely used in the art (see generally Ausubel et al., supra; Sambrook et al., supra). An isolated purified protein is generally evidenced as a single band on an SDS-PAGE gel stained with Coomassie blue.

D. Omi Antibodies

Antibodies to the Omi peptides or polypeptides or functional variants of each or functional equivalents of each are provided by the invention. Antibodies of the invention can be used, for example, to detect Omi peptides, polypeptides, variants of each or functional equivalents of each. The antibodies can be used for isolation of Omi peptides, polypeptides, variants of each or functional equivalents of each and in the identification of molecules that interact with Omi peptides, polypeptides, variants of each or functional equivalents of each. The antibodies may also be used to inhibit or enhance the biological activity of Omi peptides, polypeptides, functional variants of each or functional equivalents of each.

One such biological activity is the binding of the Omi peptides, polypeptides, functional variants of each or functional equivalents of each to at least a portion of an IAP or to a full length IAP. Preferably this portion of an IAP is at least one of the BIR domains, e.g. BIR1, BIR2 or BIR3. Accordingly, the antibodies can be specific for the N-terminus of Omi and/or inhibit the binding of the at least a portion of an IAP or the entire full length of an IAP to Omi. In one embodiment, an inhibiting antibody would be specific to an epitope that includes the amino acids Ala-Val-Pro-Ser (SEQ ID NO:3).

Within the context of the current invention, an antibody includes both polyclonal and monoclonal antibodies (mAb); Primatized™; humanized; murine; mouse-human; mouse-primate; and chimeric; and may be an intact molecule, a fragment thereof (such as scFv, Fv, Fd, Fab, Fab′ and F(ab)′₂ fragments), or multimers or aggregates of intact molecules and/or fragments; and may occur in nature or be produced, e.g., by immunization, synthesis or genetic engineering. An “antibody fragment,” as used herein, refers to fragments derived from or related to an antibody, which bind antigen and which in some embodiments may be derivatized to exhibit structural features that facilitate clearance and uptake, e.g., by the incorporation of galactose residues. This includes, e.g., F(ab), F(ab)′₂, scFv, light chain variable region (V_{L}), heavy chain variable region (V_{H}), and combinations thereof.

Antibodies are generally accepted as specific for the Omi peptides if they bind with a K_{d} of greater than or equal to 10⁻⁷M, and preferably 10⁻⁸M. The affinity of an antibody can be readily determined by one of ordinary skill in the art (see Skatchard, Ann. N.Y. Acad. Sci. 51:660-672, 1949).

Antibodies may be produced by any of a variety of methods available to one of ordinary skill in the art. Detailed methods for generating antibodies are provided in Antibodies: A Laboratory Manual, Harlow and Lane (eds.), Cold Spring Harbor Laboratories, 1988, which is incorporated by reference.

A polyclonal antibody may be readily generated in a variety of animals such as rabbits, mice and rats. Generally, an animal is immunized with a Omi peptide or one or more peptides comprising Omi amino acid sequences which may be conjugated to a carrier protein. Routes of administration include intraperitoneal, intramuscular, intraocular, or subcutaneous injections, usually in an adjuvant (e.g., Freund's complete or incomplete adjuvant).

Monoclonal antibodies may be readily generated from hybridoma cell lines using conventional techniques (see Antibodies: A Laboratory Manual, Harlow and Lane (eds.), Cold Spring Harbor Laboratories, 1988). Various immortalization techniques such as those mediated by Epstein-Barr virus or fusion to produce a hybridoma may be used. In a preferred embodiment, immortalization occurs by fusion with a myeloma cell line (e.g., NS-1 (ATCC No. TIB 18) and P3×63—Ag 8.653 (ATCC No. CRL 1580)) to create a hybridoma that secretes a monoclonal antibody.

Antibody fragments, such as Fab and Fv fragments, may be constructed, for example, by conventional enzymatic digestion or recombinant DNA techniques to yield isolated variable regions of the antibody. Within one embodiment, the genes which encode the variable region from a hybridoma producing a monoclonal antibody of interest are amplified using nucleotide primers corresponding to the variable region. Amplification products are subcloned into plasmid vectors and propagated and purified using bacteria, yeast, plant or mammalian-based expression systems. Techniques may be used to change a murine antibody to a human antibody, known familiarly as a “humanized” antibody, without altering the binding specificity of the antibody. Similar techniques may be used to change a primate antibody to a human antibody (e.g. Primatize™).

Antibodies may be assayed for immunoreactivity against the Omi peptides by any of a number of methods, including western blot, enzyme-linked immuno-sorbent assays (ELISA), countercurrent immuno-electrophoresis, radioimmunoassays, dot blot assays, sandwich assays, inhibition or competition assays, or immunoprecipitation (see U.S. Pat. Nos. 4,376,110 and 4,486,530; see also Antibodies: A Laboratory Manual, Harlow and Lane (eds.), Cold Spring Harbor Laboratory Press, 1988). Techniques for purifying antibodies are those available in the art. In certain embodiments, antibodies are purified by passing the antibodies over an affinity column to which amino acid sequences of the present invention are bound. Bound antibody is then eluted. Other purification techniques include, but are not limited to HPLC or RP-HPLC, or purification on protein A or protein G columns.

A number of therapeutically useful molecules are known in the art that comprise antigen-binding sites that are capable of exhibiting immunological binding properties of an antibody molecule. The proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment which comprises both antigen-binding sites. An “Fv” fragment can be produced by preferential proteolytic cleavage of an IgM, and on rare occasions IgG or IgA, immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (“sFv”) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

Each of the above-described molecules includes a heavy chain and a light chain CDR set, respectively interposed between a heavy chain and a light chain FR set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A “humanized” antibody refers to an antibody derived from a non-human antibody (typically murine), or derived from a chimeric antibody, that retains or substantially retains the antigen-binding properties of the parent antibody but which is less immunogenic in humans. This may be achieved by various methods, including by way of example: (a) grafting only the non-human CDRs onto human framework and constant regions (humanization), or (b) transplanting the entire non-human variable domains, but “cloaking” them with a human-like surface by replacement of surface residues (“veneering”). Such methods are disclosed, for example, in Jones et al., Nature 321:522-525, 1986; Morrison et al., Proc. Natl. Acad. Sci. 81:6851-6855, 1984; Morrison and Oi, Adv. Immunol. 44:65-92, 1988; Verhoeyer et al., Science 239:1534-1536, 1988; Padlan, Molec. Immun. 28:489-498,1991; Padlan, Molec. Immun. 31(3):169-217, 1994. In the present invention, humanized antibodies include “humanized” and “veneered” antibodies. A preferred method of humanization comprises alignment of the non-human heavy and light chain sequences to human heavy and light chain sequences, selection and replacement of the non-human framework with a human framework based on such alignment, molecular modeling to predict conformation of the humanized sequence and comparison to the conformation of the parent antibody, followed by repeated back mutation of residues in the CDR region which disturb the structure of the CDRs until the predicted conformation of the humanized sequence model closely approximates the conformation of the non-human CDRs of the parent non-human antibody.

As used herein, the terms “veneered FRs” and “recombinantly veneered FRs” refer to the selective replacement of FR residues from, e.g., a rodent heavy or light chain V region, with human FR residues in order to provide a xenogeneic molecule comprising an antigen-binding site which retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface. Davies et al. (1990) Ann. Rev. Biochem. 59:439-473. Thus, antigen binding specificity can be preserved in a humanized antibody only wherein the CDR structures, their interaction with each other, and their interaction with the rest of the V region domains are carefully maintained. By using veneering techniques, exterior (e.g., solvent-accessible) FR residues which are readily encountered by the immune system are selectively replaced with human residues to provide a hybrid molecule that comprises either a weakly immunogenic, or substantially non-immunogenic veneered surface.

The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1987), updates to the Kabat database, and other accessible U.S. and foreign databases (both nucleic acid and protein). Solvent accessibilities of V region amino acids can be deduced from the known three-dimensional structure for human and murine antibody fragments. There are two general steps in veneering a murine antigen-binding site. Initially, the FRs of the variable domains of an antibody molecule of interest are compared with corresponding FR sequences of human variable domains obtained from the above-identified sources. The most homologous human V regions are then compared residue by residue to corresponding murine amino acids. The residues in the murine FR which differ from the human counterpart are replaced by the residues present in the human moiety using recombinant techniques well known in the art. Residue switching is only carried out with moieties which are at least partially exposed (solvent accessible), and care is exercised in the replacement of amino acid residues which may have a significant effect on the tertiary structure of V region domains, such as proline, glycine and charged amino acids.

In this manner, the resultant “veneered” murine antigen-binding sites are thus designed to retain the murine CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs which are believed to influence the “canonical” tertiary structures of the CDR loops. These design criteria are then used to prepare recombinant nucleotide sequences which combine the CDRs of both the heavy and light chain of a murine antigen-binding site into human-appearing FRs that can be used to transfect mammalian cells for the expression of recombinant human antibodies which exhibit the antigen specificity of the murine antibody molecule.

E. Methods of Using Omi Nucleic Acids and Peptides or Polypeptides

Omi peptides or polypeptides can induce apoptosis by interaction with the Inhibitors of Apoptosis proteins (IAPs), as well as through their serine protease activity. Studies using the Omi peptides or polypeptides of the present application revealed that Omi is an important component of both caspase-mediated and caspase-independent apoptosis. Furthermore, these studies demonstrate that Omi peptides or polypeptides are capable of regulating or altering apoptosis. Thus, the compositions described herein, including Omi nucleic acids, peptides, and antibodies, can be used for a variety of assays and for therapeutic purposes.

1. Identification of Inhibitors and Enhancers of Caspase-Mediated Apoptotic Activity

Candidate inhibitors and enhancers may be isolated or procured from a variety of sources, such as bacteria, fungi, plants, parasites, libraries of chemicals, peptides or peptide derivatives, antibodies, and the like. Inhibitors and enhancers may also be rationally designed, based on the protein structures determined from X-ray crystallography.

Without wishing to be bound to a particular theory or held to a particular mechanism, an inhibitor may act by preventing Omi release from the mitochondria, interfering with Omi binding to an IAP, or by other mechanisms. The inhibitor may act directly or indirectly. Inhibitors may include small molecules (organic molecules), peptides, polypeptides, and antibodies, for example. In one embodiment, the inhibitors prevent apoptosis. Inhibitors should have a minimum of side effects and are preferably non-toxic.

In addition, enhancers of apoptotic activity are desirable in certain circumstances. At times, increasing apoptosis will have a therapeutic effect. For example, tumors or cells that mediate autoimmune diseases are appropriate cells for destruction. Enhancers may increase the rate or efficiency of caspase processing, increase transcription or translation, decrease proteolysis, or act through other mechanisms. As will be apparent to those skilled in the art, many of the guidelines presented above apply to the design of enhancers as well. Within the context of the present invention, Omi peptides, polypeptides or functional variants of each can act as an enhancer.

Screening assays for inhibitors and enhancers will vary according to the type of inhibitor or enhancer and the nature of the activity that is being affected. Assays may be performed in vitro or in vivo. In general, assays are designed to evaluate apoptotic pathway activation (e.g., caspase protein processing, caspase enzymatic activity, cell viability, cell morphology changes, DNA laddering, and the like). In any of the assays, a statistically significant increase or decrease compared to a proper control is indicative of enhancement or inhibition. In one embodiment, the caspase utilized for the assays is selected from the group consisting of caspase-3, caspase-7 and caspase-9. Typically, an increase in cell viability as compared to a control indicates the presence of an inhibitor, while a decrease in cell viability as compared to a control indicates the presence of an enhancer.

One in vitro assay for detecting inhibitors and enhancers of caspase-mediated apoptosis is performed by examining the effect of a candidate compound on the activation of an initiator caspase (e.g., caspase 9) or an effector caspase (e.g., caspases 3-7). Briefly, procaspase 9, an IAP, cytochrome c, dATP, and an Omi peptide, polypeptide, functional variant, or functional equivalent are provided. The processing of caspase-9 into two subunits is assayed. Alternatively, caspase-9 enzymatic activity is monitored by adding procaspase-3, procaspase-7, or other effector caspases to the reaction mix and monitoring the activation of these caspases, for example, either via subunit formation or via cleavage of a substrate (e.g., acetyl DEVD-aminomethyl coumarin (amc), lamin, PRPP, PARP, and the like). Typically, an increase in processing to subunits indicates the presence of an enhancer, while a decrease in processing to subunits indicates the presence of an inhibitor.

To facilitate detection, the protein of interest is typically labeled, for example, via in vitro translation in the presence of a labeled amino acid residue. This composition is incubated with an Omi peptide, polypeptide, functional variant, or functional equivalent in the presence or absence of a candidate inhibitor or enhancer. Either the processing of caspase-9 into two subunits or the processing or activation of a coincubated effector pro-caspase can be monitored. Caspase processing is routinely monitored either by gel electrophoresis or indirectly by monitoring caspase substrate turnover. The two subunits and caspase substrate turnover may be readily detected by autoradiography after gel electrophoresis. One skilled in the art will recognize that other methods of labeling and detection may be used alternatively.

Moreover, any known enzymatic analysis can be used to follow the inhibitory or enhancing ability of a candidate compound with regard to the ability of Omi peptide of the present invention, or variants thereof, to promote the enzymatic activity of caspases. For example, one can express an Omi construct of interest in a cell line, be it bacterial, insect, mammalian, or other, and purify the resulting polypeptide. The purified Omi peptide can then be used in a variety of assays to follow its ability to promote the enzymatic activity of effector caspases or apoptotic activity. Such methods of expressing and purifying recombinant proteins are known in the art, and examples can be found in Sambrook et al., Molecular Cloning: A Laboratory Manual, Cold Spring Harbor Press, 1989 as well as in a number of other sources. Typically, an increase in caspase enzymatic activity indicates the presence of an enhancer, while a decrease in caspase enzymatic activity indicates the presence of an inhibitor.

In vivo assays are typically performed in cells transfected either transiently or stably with an expression vector containing an Omi nucleic acid molecule such as those described herein. These cells may be used to measure caspase processing, caspase substrate turnover, enzymatic activity of effector caspases, or apoptosis in the presence or absence of a candidate compound. When assaying apoptosis, a variety of methods of cell analyses may be used, including, for example, dye staining and microscopy to examine cell viability, nucleic acid fragmentation, porosity of the cells, and membrane blebbing.

The functional activities of proteins and polypeptides are often regulated via physical interactions with other proteins or polypeptides. Such protein:protein interactions may directly or indirectly effect protein function. For example, subcellular localization, half-life and stability, conformation, and functional interactions with other molecules may all be affected by a protein's binding partners. The present invention discloses that Omi binds IAP proteins. In addition, it is demonstrated that Omi apoptosis-inducing activities are mediated by interaction with an IAP or serine protease substrate. Since many proteins operate within the context of a larger functional complex, it is likely that Omi also interacts with other molecules. Thus, the present invention provides methods of identifying Omi binding molecules, and methods for identifying compounds that inhibit or enhance Omi binding to an Omi-binding molecule. Omi binding molecules may potentially enhance or inhibit Omi's activities, including Omi's ability to induce both caspase-independent and caspase-mediated apoptosis. Therefore, molecules that inhibit or enhance Omi binding to an Omi-binding molecule may, themselves, indirectly enhance or inhibit an Omi functional activity.

Methods for evaluating the ability of a candidate compound to inhibit or enhance Omni binding to an Omi-binding molecule are well known to one of ordinary skill in the art. Such assays are generally performed utilizing standard binding assays for measuring binding of an Omi-binding molecule to Omi, such as those disclosed supra. A candidate compound may be added to the binding reaction, and its ability to inhibit or enhance Omni binding to an Omi-binding molecule is determined by comparing binding in the presence or absence of a candidate compound. In certain circumstances, such assays will detect displacement or inhibition of binding of an Omi-binding molecule from an Omi peptide or polypeptide. Candidate compounds may be obtained from a variety of sources. For example, they may be produced recombinantly, purified from a natural source, or synthesized. Preferably, candidate compounds will be at least partially purified, and most preferably, candidate compounds will be purified to near homogeneity. Appropriate controls depend upon the source of the candidate compound and might include, for example, extracts from cells transfected with control vector, unprogrammed reticulocyte lysate, or storage or reaction buffers. Alternatively, functional assays may be performed to identify a compound that inhibits or enhances Omi binding to an Omi-binding molecule. One example of such a functional assay is to contact an Omi peptide or polypeptide in the presence of an Omi-binding molecule with an initiator and/or effector caspase, cytochrome c, and ATP or dATP. The effect of the candidate compound is determined by determining the levels of caspase processing activity. A decrease in processing shows the candidate compound disrupted binding of Omi to an Omi-binding molecule, while an increase in processing shows the candidate compound stabilized binding of Omi to an Omi-binding molecule. Another example is a functional assay that detects the presence of a substrate cleavage produced by caspase cleavage of a substrate. Preferred methods of the invention utilize caspase-9 as the initiator caspase and either caspase-3 or caspase-7 as the effector procaspase. Preferred methods utilize acetyl DEVD-aminomethyl coumarin as the cleavage substrate.

A variety of methodologies exist that can be used to investigate the effect of a candidate compound. Such methodologies are those commonly used to analyze enzymatic reactions and include, for example, SDS-PAGE, spectroscopy, HPLC analysis, autoradiography, chemiluminescence, chromogenic reactions, and immunochemistry (e.g., blotting, precipitating, etc.).

2. Compositions and Methods of Modulating Apoptosis

Compositions comprising an Omi peptide, polypeptide, functional variant, as defined above, or antibody are provided by the invention. Such compositions may be used to inhibit or promote apoptosis. In certain embodiments, compositions comprise a nucleic acid molecule of the present invention, a peptide of the present invention, or an antibody of the present invention; and a physiologically acceptable carrier. In certain embodiments, compositions comprise compounds for inhibiting or enhancing caspase-dependent apoptosis in a cell, where such compounds are identified based on their ability to inhibit or enhance Omi-mediated apoptosis or their ability to disrupt or stabilize Omi binding to an Omi-binding polypeptide.

Antibodies of the invention include, but are not limited to, polyclonal, monoclonal, single chain, or humanized antibodies or antibody fragments. Compositions of the invention comprise, for example, polyclonal antibodies that recognize one or more epitopes of Omi, particularly on the N-terminus. In one embodiment, an antibody recognizes an epitope that includes the amino acids Ala-Val-Pro-Ser (SEQ ID NO:3). In another embodiment, an antibody recognizes an epitope within the serine protease domain of Omi, said domain including the amino acids Gly-Asn-Ser-Gly-Gly-Pro-Leu (SEQ ID NO:11). Alternatively, they can comprise monoclonal antibodies that recognize other specific epitopes of Omi. The antibodies of the composition may recognize native Omi and/or denatured Omi. These antibodies may be produced according to methods well known in the art, as described above.

Examples of polynucleotide compositions include mammalian expression vectors, sense RNAs, ribozymes, and antisense RNA. Expression vectors and sense RNA molecules are designed to express Omi peptides or polypeptides, while ribozymes and antisense RNA constructs are designed to reduce the levels of Omi expressed.

The compositions may also contain a physiologically acceptable carrier. The term “physiologically acceptable carrier” refers to a carrier for administration of a first component of the composition that is selected from antibodies, peptides, polypeptides, or nucleic acids. Suitable carriers and physiologically acceptable salts are well known to those of ordinary skill in the art. A thorough discussion of acceptable carriers is available in Remington's Pharmaceutical Sciences, Mack Publishing Co., NJ, 1991).

Appropriate dosage amounts balancing toxicity and efficacy will be determined during any clinic testing pursued, but a typical dosage will be from about 0.001 mg/kg to 50 mg/kg or 0.05 mg/kg to about 10 mg/kg of the polynucleotide, peptide or antibody. If used in gene therapies such dosages will depend on the vector utilized and will be determined during clinical testing.

The compositions of the invention can be (1) administered directly to the subject; (2) delivered ex vivo to cells derived from the subject; or (3) delivered in vitro. Direct delivery will generally be accomplished by injection. Alternatively, compositions can also be delivered via oral or pulmonary administration, suppositories, transdermally, or by gene guns, for example. Dosage treatment may be a single dose or multiple doses.

Methods of ex vivo delivery and reimplantation of transformed cells into a subject are known in the art. Generally, delivery of nucleic acids for both ex vivo and in vitro applications can be accomplished by, for example, dextran-mediated transfection, calcium phosphate precipitation transfection, viral infection, polybrene mediated transfection, protoplast fusion, electroporation, encapsulation of polynucleotides in liposomes, and direct microinjection of the DNA into nuclei, all well known in the art.

Gene therapy vectors comprising Omi nucleic acid sequences, or complements or variants thereof, are within the scope of the invention. These vectors may be used to regulate Omi mRNA and peptide or polypeptide expression in target cells. In some instances, it may be advantageous to increase the amount of Omi nucleic acids or Omi peptides or polypeptides that are expressed. In other cases, gene therapy vectors may be used to decrease functional Omi levels. Gene therapy vectors may comprise any Omi nucleic acid of the current invention, including fragments, variants, antisense, ribozymes, and mutants. Expression of Omi nucleic acids may be controlled by endogenous mammalian or heterologous promoters and may be either constitutive or regulated. Nucleic acids used according to the invention may be stably integrated into the genome of the cell or may be maintained in the cell as separate episomal segments of DNA.

Omi nucleic acid molecules may be delivered by any method of gene delivery available in the art. A gene delivery vehicle may be of viral or non-viral origin (see generally Jolly, Cancer Gene Therapy 1:51-64, 1994; Kimura, Human Gene Therapy 5:845-852, 1994; Connelly, Human Gene Therapy 1:185-193,1995; and Kaplitt, Nature Genetics 6:148-153, 1994). The present invention can employ recombinant retroviruses which are constructed to carry or express a Omi nucleic acid molecule. Methods of producing recombinant retroviral virions suitable for gene therapy have been extensively described (see, e.g., Mann et al. Cell 33:153-159, 1983; Nikolas and Rubenstein, Vectors: A survey of molecular cloning vectors and their uses, Rodriquez and Denhardt (eds.), Stoneham:Butterworth, 494-513, 1988).

The present invention also employs viruses such as alphavirus-based vectors, adenovirus, and parvovirus that can function as gene delivery vehicles. Examples of vectors utilized by the invention include intact adenovirus, replication-defective adenovirus vectors requiring a helper plasmid or virus, and adenovirus vectors with their native tropism modified or ablated such as adenoviral vectors containing a targeting ligand. Other examples include adeno-associated virus based vectors and lentivirus vectors.

Packaging cell lines suitable for use with the above-described viral and retroviral vector constructs may be readily prepared and used to create producer cell lines (also termed vector cell lines) for the production of recombinant vector particles.

Examples of non-viral methods of gene delivery vehicles and methods which may be employed according to the invention include liposomes (see, e.g., Wang et al. PNAS 84:7851-7855, 1987), polycationic condensed DNA (see, e.g., Curiel, Hum. Gene Ther. 3:147-154, 1992); ligand linked DNA (see, e.g., Wu, J. Biol. Chem. 264:16985-16987, 1989); deposition of photopolymerized hydrogel materials; hand-held gene transfer particle guns, as described in U.S. Pat. No. 5,149,655; ionizing radiation as described in U.S. Pat. No. 5,206,152 and WO 92/11033; and nucleic charge neutralization or fusion with cell membranes. Additional approaches are described in Philip, Mol. Cell Biol. 14:2411-2418, 1994 and in Woffendin, Proc. Nat. Acad. Sci. 91:1581-1585, 1994. Conjugates comprising a receptor-binding internalized ligand capable of delivering nucleic acids may also be used according to the present invention. Conjugate-based preparations and methods of use thereof are described in WO 96/36362, which is hereby incorporated by reference in its entirety. Other non-viral delivery methods include, but are not limited to, mechanical delivery systems such as the approach described in Woffendin et al., Proc. Natl. Acad. Sci. USA 91(24):11581-11585, 1994 and naked DNA protocols. Exemplary naked DNA introduction methods are described in WO 90/11092 and U.S. Pat. No. 5,580,859.

In other embodiments, methods of the invention utilize bacteriophage delivery systems capable of transfecting eukaryotic cells. Bacteriophage-mediated gene transfer systems are described in WO 99/10014, which is incorporated in its entirety. Phage delivery vehicles may express a targeting ligand on their surface that facilitates receptor-mediated gene delivery.

In addition, compositions and methods of modulating apoptosis using small molecule agonists or antagonists or heterologous polypeptides that bind Omi are included within the scope of the current invention. Similarly, compositions comprising small molecule agonists or antagonists or heterologous polypeptides that enhance or inhibit Omi-mediated apoptosis are included in the invention. Compositions comprising small molecules or heterologous polypeptides that disrupt or stabilize Omi binding to another molecule are also included within the scope of the current invention.

Compositions of the invention may be used to inhibit or promote apoptosis both in vitro and in vivo. In certain embodiments, compositions of the invention are pharmaceutical compositions for the treatment of diseases associated with undesirable cell proliferation, such as, for example, cancer and autoimmune diseases. In other embodiments, compositions of the invention are pharmaceutical compositions for the treatment of diseases associated with inappropriate activation of apoptosis, such as, for example, neurodegenerative diseases and ischemic injury. Pharmaceutical compositions of the invention comprising compounds that inhibit or enhance Omi-mediated apoptosis can be produced by identifying such a compound, as described supra, and purifying the compound to a degree necessary to ensure safety and efficacy, such parameters determined by routine pharmaceutical and clinical practice. Quantities of the compound may be produced, for example, by recombinant expression in a suitable host cell or chemical synthesis, prior to purification.

### EXAMPLES

The following experimental examples are offered by way of illustration, not limitation.

### Example 1

### Identification of Additional BIR3 Domain Binding Proteins

This example discloses the identification of human proteins that bind to the Smac and caspase-9 binding pocket on the BIR3 domain of human XIAP. Specifically, the serine protease Omi (also known as HtrA2) was identified and isolated.

As mentioned above, the mechanism of IAP inhibition of caspases is conserved in mammals and insects. Accordingly, it is thought that other mammalian IAP-binding proteins are still undiscovered. To identify such proteins, a GST-BIR3 fusion protein was used as an affinity reagent to purify new IAP binding proteins from extracts of human HEK293 cells and mouse heart, kidney, liver and spleen tissues. Five milliliters of a 293 cell pellet was washed once in phosphate buffered saline (10 mM phosphate, pH 7.4, containing 150 mM NaCl), centrifuged and the pellet was re-suspended in ten milliliters of cell lysis buffer (20 mM Tris-HCl, pH 7.4, 150 mM NaCl, 1 mM dithiothreitol, 1 mM EDTA, 1 mM EGTA supplemented with protease inhibitor mix (Sigma P-8340) containing AEBSF, pepstatin A, E-64, bestatin, leupeptin and aprotinin). Cells were homogenized and centrifuged once at 20,000×g and the supernatant was then subjected to centrifugation at 100,000×g. The resultant supernatant was pre-cleared with Glutathion-Sepharose beads and then incubated with GST-BIR3 bound to Glutathion-Sepharose beads for 12 hours at 4° C. The mixture was centrifuged at 500 rpm for three minutes and the GST-BIR3 bead pellet was washed three times in the lysis buffer. After binding the extracts to the GST-BIR3 protein, the bound proteins were eluted with 200 μM of the Smac IAP-binding motif peptide AVPIA (SEQ ID NO:2) at 37° C. for one hour and analyzed by far-western blotting with ³⁵S-labeled in vitro translated XIAP. Specifically, the affinity-purified proteins were subjected to SDS-PAGE electrophoresis and then transferred to a nitrocellulose membrane using standard western blotting technique. The proteins on the membrane were probed with ³⁵S-labeled in vitro translated XIAP as described in Srinivasula, S. M. et al., Nature 410:112-116, 2001.

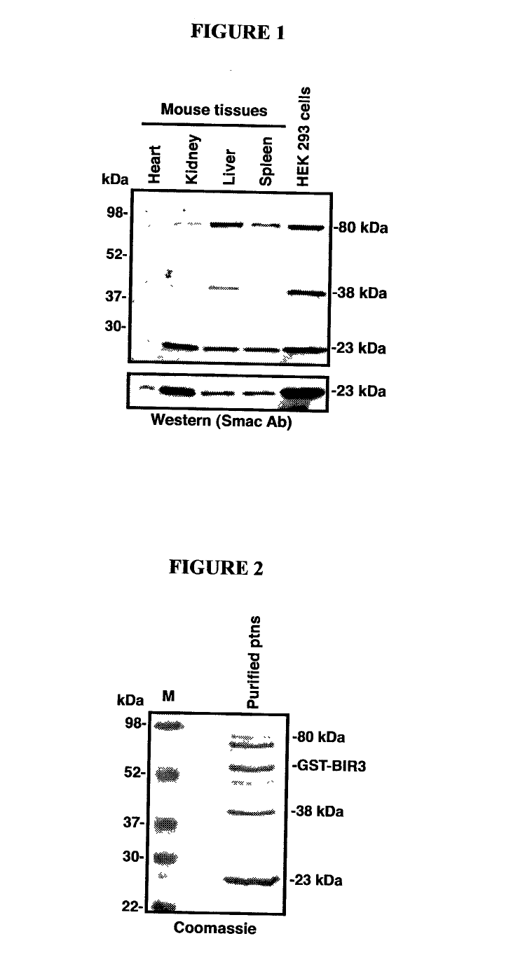

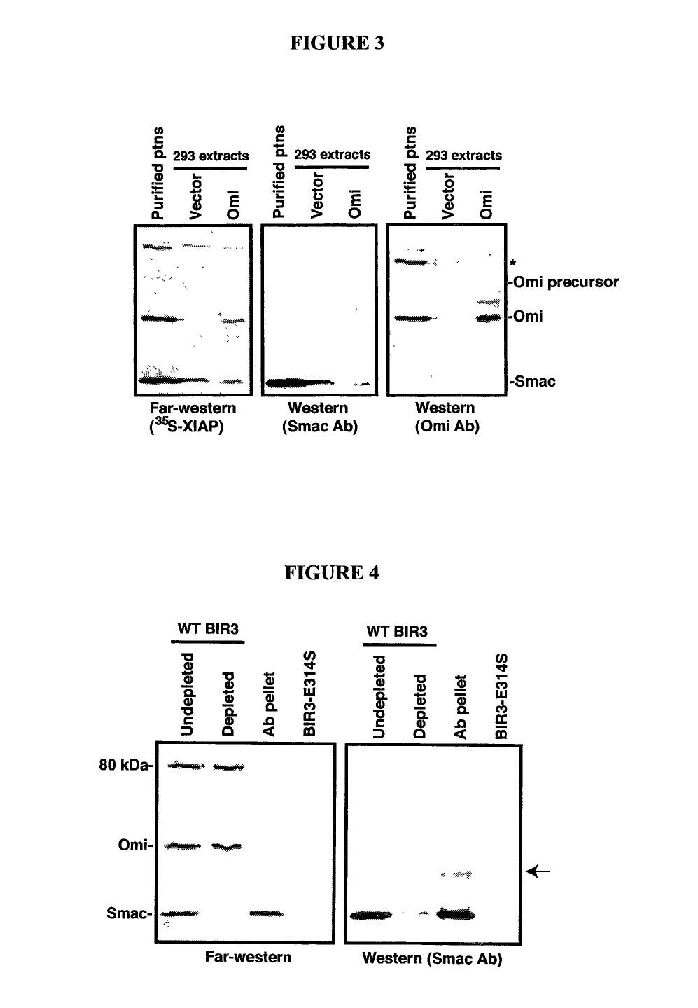

Three proteins, migrating as 23, 38 and 80 kDa bands, that specifically interact with the ³⁵S-labeled XIAP were detected in the AVPIA-peptide eluates from the human 293 cells and the mouse tissues (FIG. 1). A large-scale affinity purification was then fractionated by SDS-PAGE (FIG. 2) and Coomassie stained bands were cut and subjected to microsequencing. Due to the low level of the 80 kDa protein in this preparation, limited N-terminal sequence information was obtained, which was not sufficient for protein identification. The N-terminal sequence information of the other two proteins revealed that the 23 kDa protein is mature Smac/Diablo and the 38 kDa band is a previously described serine protease named Omi or HtrA2 (GenBank Accession number XM_{—}002750; SEQ ID NO:1) (Faccio, L. et al., J Biol Chem 275:2581-2588, 2000; Gray, C. W. et al., Eur J Biochem 267:5699-5710, 2000). Mass spectrometric peptide fingerprinting of these two protein bands confirmed the microsequencing results. The band immediately below the 80 kDa band was identified as Hsp70 (see FIG. 2). Furthermore, western blot analysis of the AVPIA-peptide eluates using Smac and Omi antibodies confirmed that the 23 kDa band represents Smac and the 38 kDa band represents Omi (FIGS. 1 and 3).

Regarding FIG. 3, 293-T cells were transfected with an Omi precursor expression construct (Omi lane) or empty vector (vector lane) and their extracts were fractionated by SDS-PAGE and then far-western blotted with ³⁵S-labeled XIAP (left panel) or immunoblotted with Smac antibody (middle panel) or an Omi polyclonal antibody (right panel). The first lane of each of panel of FIG. 3 represents the GST-BIR3-affinity purified proteins from 293 cells. A small amount of the unprocessed Omi precursor protein can be seen in the Omi lane.

To confirm that Omi associates directly with the BIR3 domain of XIAP but not with the Smac protein, Smac was depleted from the 293 extracts using a Smac monoclonal antibody. Specifically, 293 cellular extracts were either left untreated (undepleted lane of FIG. 4) or depleted with a Smac antibody (lane depleted of FIG. 4). The undepleted and depleted extracts were incubated with the GST-BIR3 protein and the bound proteins were eluted as above. The eluates were then analyzed by far-western blotting with ³⁵S-XIAP and by western blotting with the Smac antibody as described above (see FIG. 4, left and right panels, respectively). Depletion of Smac did not remove Omi from the 293 extracts, indicating that Omi binds independently to the GST-BIR3 protein. The last lane of each panel in FIG. 4 represents the 293 cellular extracts that were bound to the E314S mutant GST-BIR3 fusion protein. This mutation has been shown to abolish binding of Smac and caspase-9 to BIR3. These results indicate that Omi, Smac and the 80 kDa protein associate directly with the Smac binding pocket on the BIR3 of XIAP. The asterisk in FIG. 4 indicates non-specific bands and the arrow indicates the IgG band.

### Example 2

### OMI'S BIR3 Binding Activity Resides in its N-Terminus

This example discloses that Omi is a bona fide IAP binding protein with an N-terminal IAP binding motif essential for its ability to bind IAPs. It was discovered that the BIR3-affinity purified Omi started with an amino-terminal AVPS sequence (SEQ ID NO:3), which appears to represent a conserved IAP-binding motif.

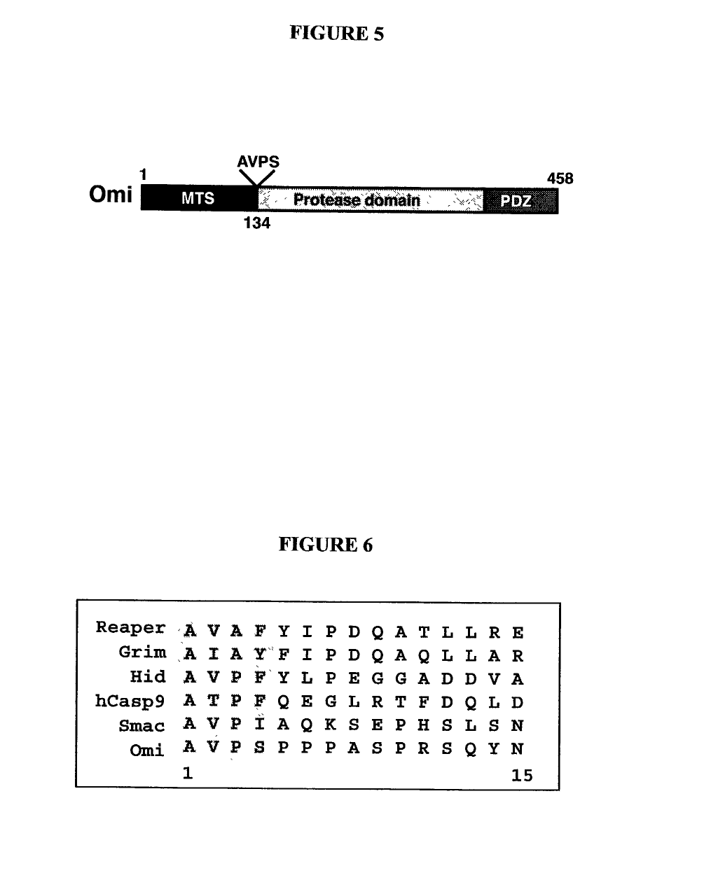

FIG. 6 is a collinear alignment of the N-terminal sequences of Drosophila Reaper (SEQ ID NO:4), Grim (SEQ ID NO:5) and Hid (SEQ ID NO:6) and human caspase-9-p12 (SEQ ID NO:7), Smac (SEQ ID NO:8) and Omi (SEQ ID NO:9). Based on the deduced amino acid sequence of the cloned Omi protein, this IAP binding motif starts at residue 134, indicating that Omi is made as a precursor protein that undergoes proteolytic processing at residue 133 to remove the N-terminal leader sequence. FIG. 5 provides a bar diagram representation of the structure of the Omi precursor and the location of the IAP binding motif.

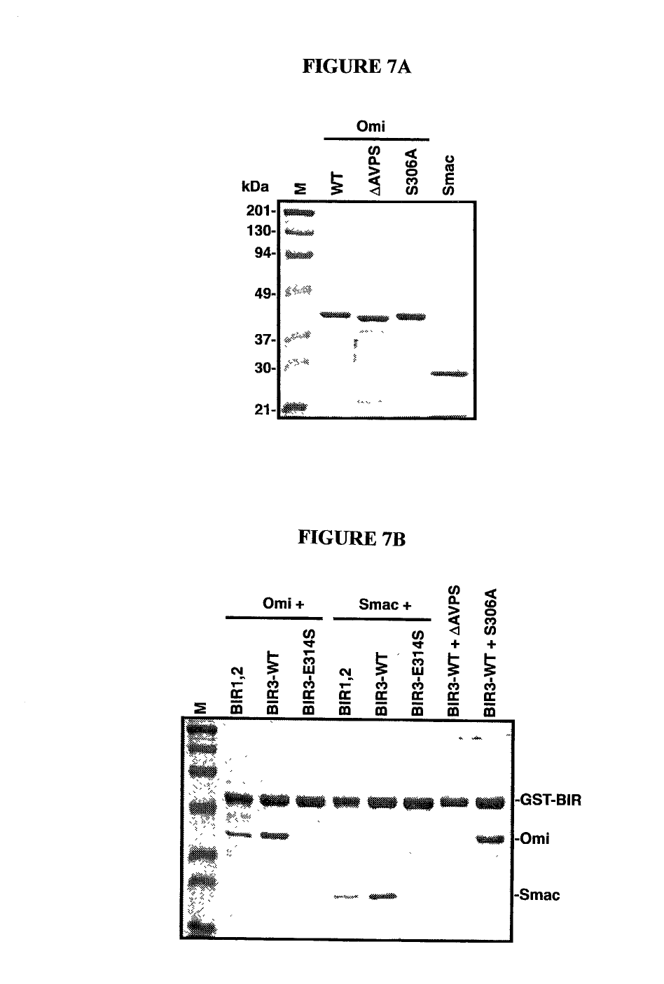

To determine whether a recombinant Omi lacking the first 133 residues (mature Omi) can interact with the GST-BIR3 fusion protein, this protein was expressed with a C-terminal His6 tag in bacteria and purified to apparent homogeneity (see FIG. 7B). N-terminal amino acid sequencing of the recombinant proteins indicated that the initiator methionine is removed. FIGS. 7A and 7B show scanned images of Coomassie stained gels representing SDS-PAGE analysis of purified C-terminal His6-tagged recombinant wild type mature Omi (WT), Omi-ΔAVPS, Omi-S306A, and mature Smac (FIG. 7A) and recombinant Omi and Smac proteins with GST-BIR1, 2, GST-BIR3 or GST-BIR3-E314S fusion proteins (FIG. 7B). Both recombinant mature Omi and Smac were able to interact equally with the wild type GST-BIR3 protein but not with the GST-BIR3-E314S mutant (FIG. 7A). Mature Omi and Smac were also able to interact with the GST-BIR1, 2 fusion protein. Mutation of the active site Ser306 to Ala of mature Omi did not affect its interaction with GST-BIR3. However, deletion of the AVPS motif of mature Omi completely abolished its interaction with the GST-BIR3 fusion protein.

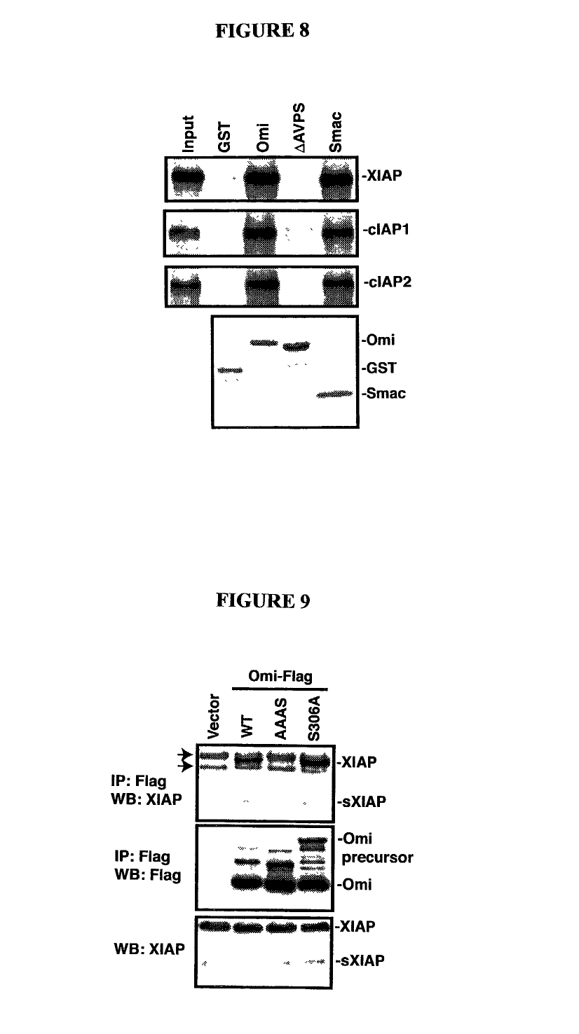

FIG. 8 shows the in vitro interaction of XIAP, cIAP1 and cIAP2 with mature Omi and Smac. C-terminal His6-tagged mature Omi or Smac, or a GST protein were immobilized onto Talon-affinity resin. The bound resins were incubated with in vitro-translated ³⁵S-labeled XIAP (first panel), cIAP1 (second panel) or cIAP2 (third panel), washed extensively, and then analyzed by SDS polyacrylamide gel electrophoresis and autoradiography. GST was used as a negative control (second lane of each panel). The fourth panel, represents a scanned image of a Coomassie stained gel of the immobilized proteins. Mature Omi was found to also interact with cIAP1 and cIAP2. The in vitro assays were performed as described in Srinivasula, S. M. et al., J Biol Chem 275:36152-36157, 2000.

Mature Omi can also interact with endogenous XIAP in vivo (see FIG. 9). This interaction requires the AVPS motif as mutation of this motif to AAAS abolished its interaction with endogenous XIAP (FIG. 9, lane 3 of first panel). Interestingly, mutation of the AVPS motif to AAAS did not affect processing and removal of the N-terminal leader sequence of the Omi precursor (FIG. 9, second panel). The above data indicate that Omi is a bona fide IAP binding protein with an N-terminal IAP binding motif essential for its ability to bind IAPs.

### Example 3

### OMI Colocalizes with SMAC in the Inter-Mitochondral Membrane Space

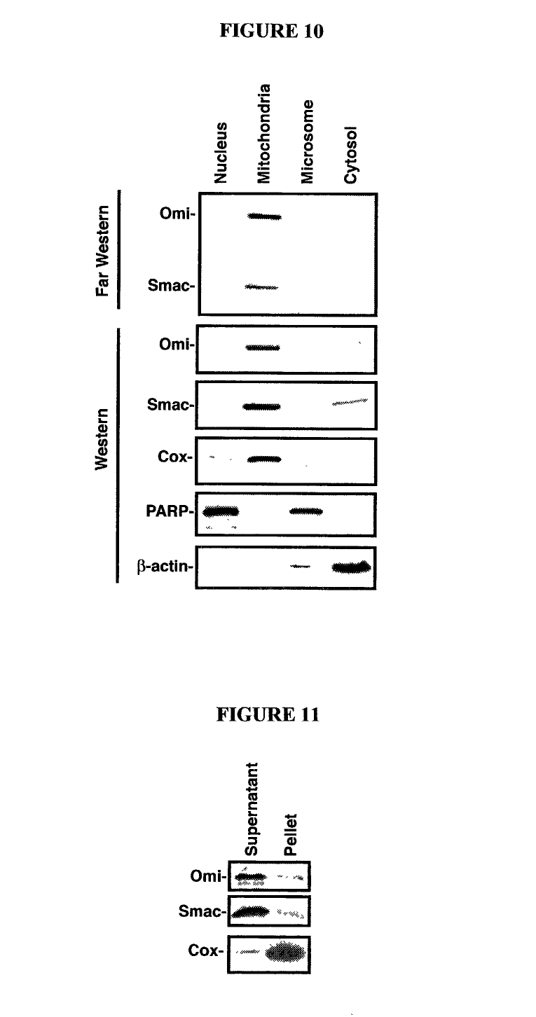

Computer analysis using the PSORT program revealed that the N-terminal leader sequence of the Omi precursor contains within its first 60 residues a typical mitochondrial targeting sequence. This sequence might be removed by mitochondrial processing peptidases upon import into the mitochondria. To examine whether the endogenous mature Omi protein is indeed localized in the mitochondria, 293 cells were subfractionated into cytosolic, mitochondrial, microsomal and nuclear fractions. Each fraction was then applied to see if it binds to a GST-BIR3 protein. Cells were homogenized in Buffer A (20 mM Hepes, pH 7.5,10 mM KCl, 1.5 mM MgCl2, 1 mM EDTA, 1 mM EGTA, 250 mM sucrose, 1 mM DTT, 0.1 mM PMSF, with protease inhibitor mix). The homogenate was centrifuged at 800×g. Nuclei were prepared from the crude nuclear pellet as described in Martin, S. J. et al., Embo J 14:5191-5200, 1995. The supernatant was centrifuged at 10,000×g, and the resultant pellet was further processed for mitochondria purification over a percoll gradient. The post-mitochondrial supernatant was subjected to 100,000×g and the microsomal pellet and cytosolic supernatants were collected separately. As shown in FIG. 10, far-western with ³⁵S-XIAP (first panel) and western blot analyses with Omi and Smac antibodies (second and third panels, respectively) showed the majority of Omi and Smac to be localized in the mitochondrial fraction. Total subcellular fractions were also immunoblotted with antibodies against Cytochrome c oxidase (Cox) (FIG. 10, fourth panel), PARP (FIG. 10, fifth panel) and β-actin (FIG. 10, sixth panel).

Next purified mitochondria from HEK293 cells were subfractionated into inter-membrane space and matrix fractions using a buffer containing 1-1.2% digitonin, which specifically disrupts the outer mitochondrial membrane without affecting the inner mitochondrial membrane. Mitoplasts (pellet) were then analyzed by western blotting with Omi (FIG. 11, upper panel), Smac (FIG. 11, middle panel) and cytochrome c oxidase (FIG. 11, lower panel) antibodies. Both Omi and Smac were predominantly present in the inter-membrane space fraction.

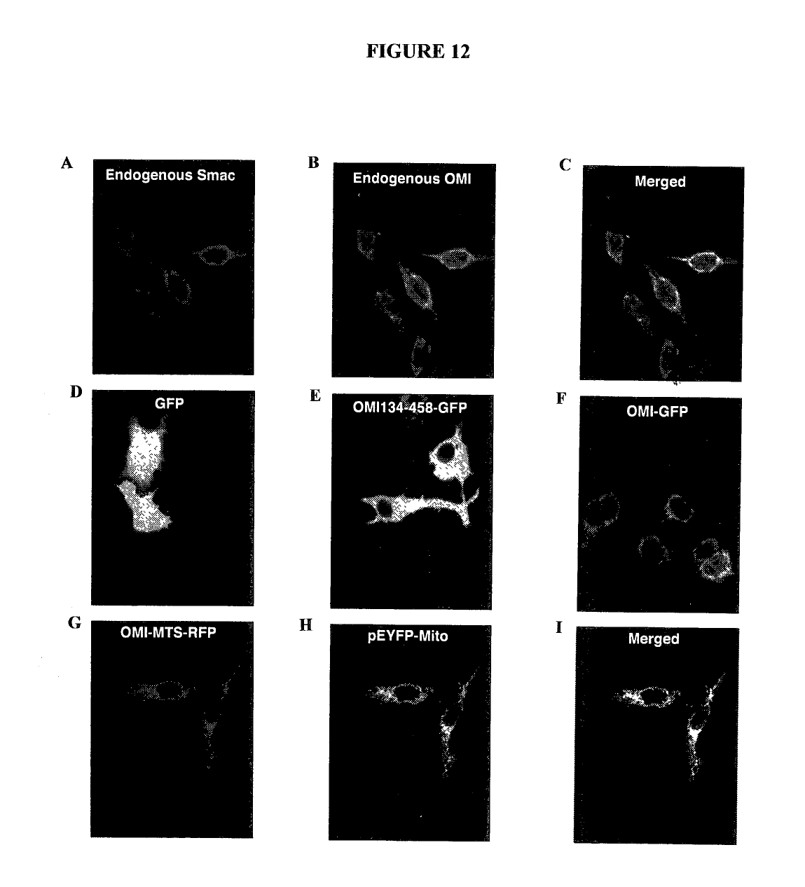

To confirm the mitochondrial localization of Omi, cells with C-terminal GFP-tagged Omi precursor and RFP-tagged Smac precursor were co-transfected. FIGS. 12A-12I represent confocal micrographs of MCF-7 cells transfected with C-terminal red fluorescence protein-tagged Smac precursor (Smac-RFP, 12A), green fluorescence protein-tagged Omi precursor (Omi-GFP, 12B), GFP (12D), C-terminal GFP-tagged mature Omi (Omi134-458-GFP, 12E) or Omi precursor (Omi-GFP, 12F), C-terminal RFP-tagged Omi 1-60 (Omi-MTS-RFP, 12G) and pEYFP-Mito marker (12H). For the immunofluorescence confocal microscopy, cells were grown on cover slips and then stained with a polyclonal antibody raised against pure mature recombinant Omi protein, and a mouse anti-Smac monoclonal antibody after fixing the cells with 4% paraformaldehyde. FITC conjugated anti rabbit and Rhodamine conjugated anti mouse antibodies were used as secondary antibodies. After staining, the cover slips were mounted on Iside and observed using confocal microscopy. The right panels in FIGS. 12C and 12F represent merged micrographs schematic diagram of a GFP-Omi fusion protein and its cleavage by caspase-8 to generate a mature cytosolic Omi. Both the GFP-tagged Omi and the RFP-tagged Smac exhibited similar pattern of punctate staining, indicative of mitochondrial localization (see FIGS. 12A-12C). Removal of the mitochondrial targeting sequence of Omi resulted in expression of mature Omi (residues 134-458) in the cytoplasm (see FIGS. 12D-12F). The first 60 residues of the Omi precursor, which harbors the MTS, was sufficient for targeting RFP to the mitochondria when expressed as a fusion protein with RFP (see FIG. 12G-12I). Taken together, the above data indicate that Omi co-localizes with Smac in the inter-mitochondrial membrane space.

### Example 4

### OMI N-Terminal Peptides Promote Caspase Activation

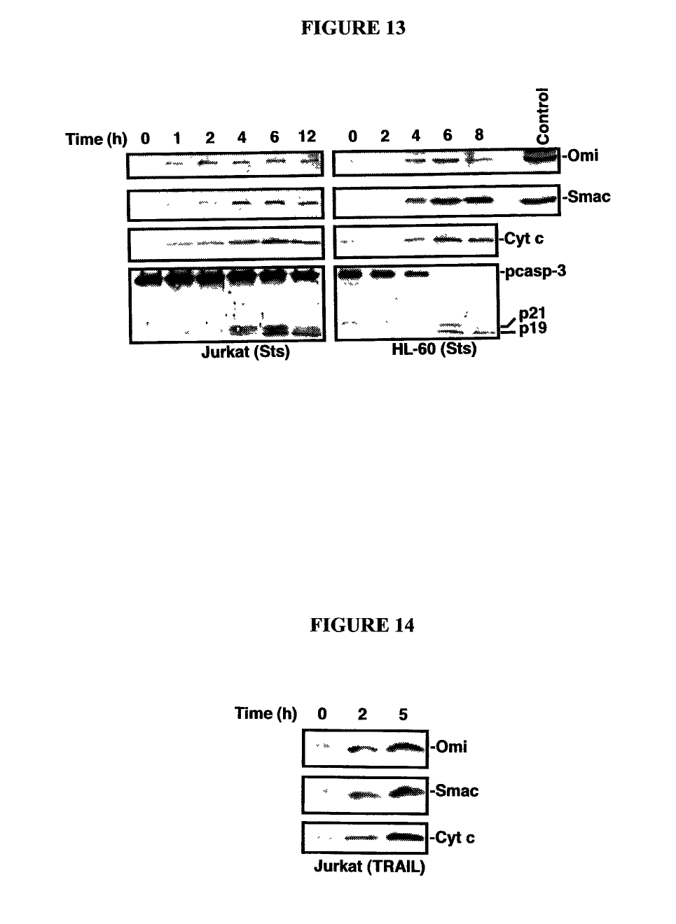

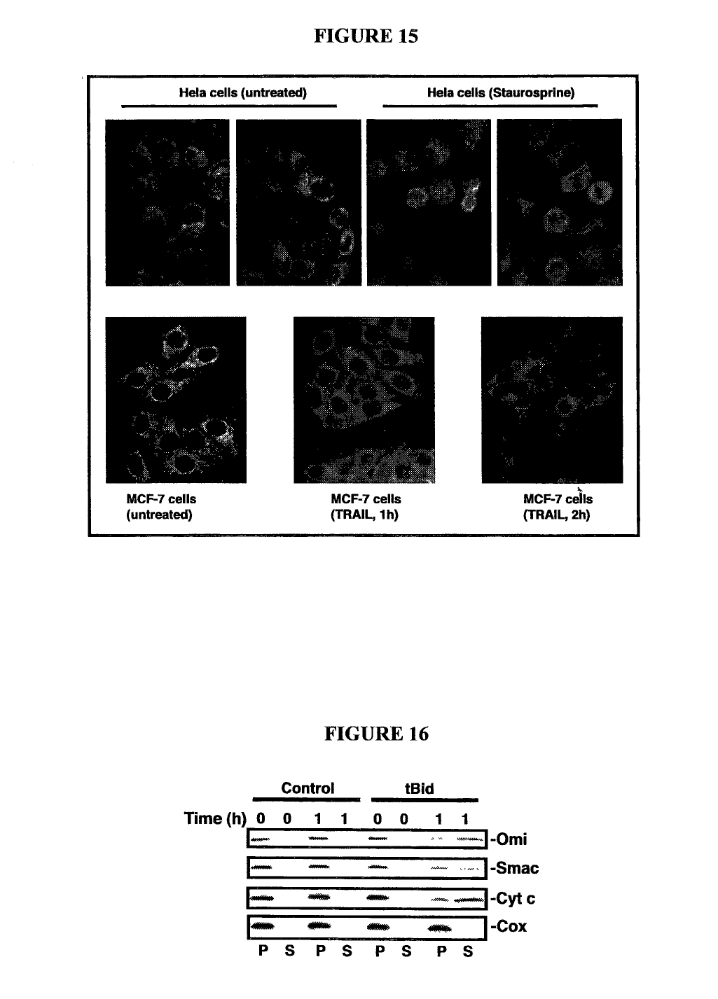

To determine whether Omi is released together with Smac and cytochrome c from the mitochondria to the cytoplasm during apoptosis, we treated Jurkat and HL-60 cells with staurosporine (2 μM), which is known to induce the mitochondrial apoptotic pathway, and analyzed their cytosolic extracts by immunoblot analysis. As shown in FIG. 13, cytochrome c, Smac, and Omi accumulated in the cytosol of these cells in a time dependent manner after treatment with staurosporine. Processing of procaspase-3 also followed a similar time course with maximum processing observed at 6-8 hours after treatment. Very little or no Omi or Smac proteins or processing of procaspase-3 were detected in the extracts at zero-time points. Similar results were obtained after treatment of Jurkat cells with TRAIL (see FIG. 14). Immunofluorescence confocal microscopy of staurosporine-treated Hela cells and TRAIL-treated MCF-7 cells showed diffused immunofluorescence staining of Omi, whereas in the untreated control cells it is mostly punctate and perinuclear (see FIG. 15). The above data clearly show that Omi is released together with cytochrome c and Smac during apoptosis.

The ability of TRAIL to induce release of Omi from the mitochondria of MCF-7 and Jurkat cells indicate that tBid, which is generated by active caspase-8 after TRAIL-receptor ligation, is responsible for the release of Omi. To test this hypothesis, mitochondria were isolated and incubated with a physiological amount of tBid. As shown in FIG. 16, tBid induced release of Omi, Smac and cytochrome c from the mitochondria into the supernatants. Combined, the above data clearly show that Omi is released from the mitochondria together with cytochrome c and Smac during apoptosis and after stimulation of mitochondria with tBid.

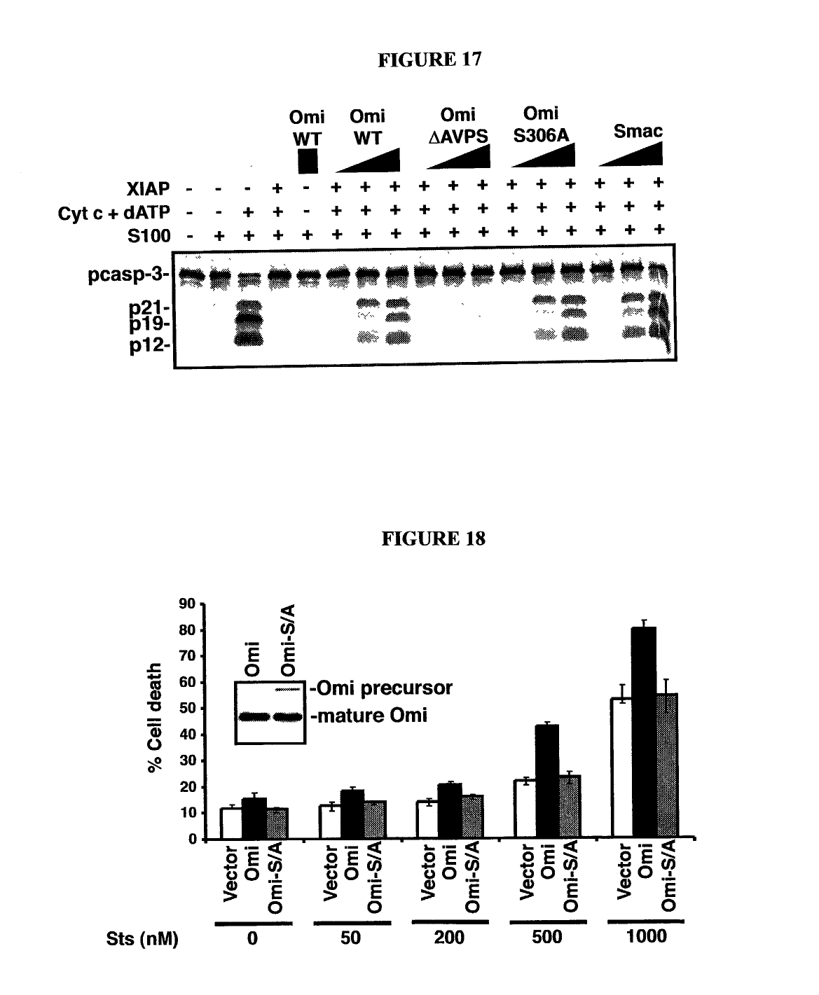

Next, it was determined whether Omi, like Smac, promotes caspase-9 activity in HEK293 S100 extracts in the presence of XIAP. To measure the caspase-9 activity in these extracts, ³⁵S-labeled procaspase-3 was added to the S100 extracts and stimulated the extracts with cytochrome c and dATP. As shown in FIG. 17, mature WT Omi or the S306A mutant were able to promote procaspase-3 activation in the XIAP-containing S100 extracts almost to the same extent as Smac. The ability to potentiate procaspase-3 activation was dependent on the AVPS motif of Omi, since deletion of this motif abolished its caspase-promoting activity. These observations indicate that Omi, like Smac, is able to disrupt the interaction of caspase-9 with XIAP in the S100 extracts to promote procaspase-3 activation.

### Example 5

### OMI Induces Apoptosis via its Serine Protesase Activity and Binding to IAPs

This example discloses that Omi can induce apoptosis in mammalian cells independent of caspases, Apaf-1, or IAPs via its serine protease activity. To determine whether release of Omi can potentiate apoptosis, human Hela cells were transfected with the Omi precursor, and the cells were stimulated with different doses of staurosporine. It is reasoned that treatment with staurosporine should release the overexpressed Omi from the mitochondria, thereby enhancing apoptosis. As shown in FIG. 18, transiently expressed Omi did not induce apoptosis in the absence of staurosporine. However, at 500 nM of staurosporine, ˜43% of the Omi-transfected cells showed signs of apoptosis compared to ˜20% in the case of the vector transfected cells. With higher concentrations of staurosporine increased apoptosis was observed in the Omi transfected cells compared to the vector-transfected cells. No apoptosis potentiation was observed with an active site Omi precursor mutant, which also has a mutant VVAS sequence instead of the AVPS motif. These observations indicate that release of Omi by an apoptotic stimulus such as staurosporine enhances cell death.

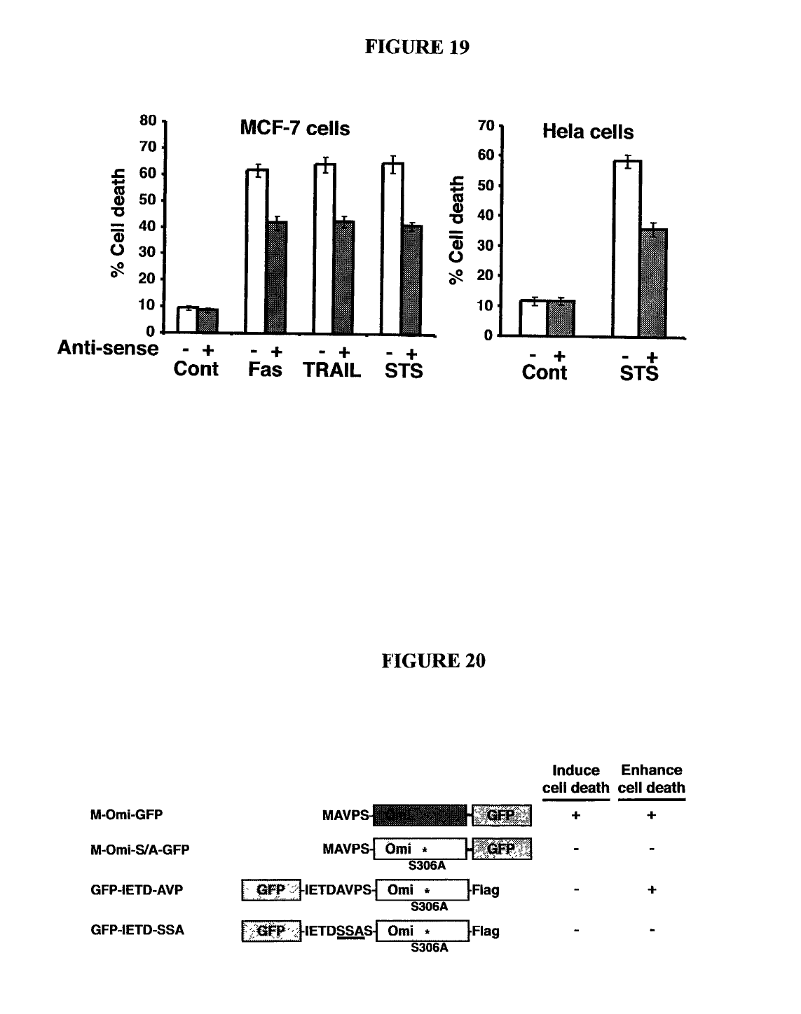

To determine if endogenous Omi plays a role in cell death, MCF-7 and Hela cells were transfected with full-length anti-sense Omi cDNA to reduce the expression of Omi in the transfected cells. MCF-7 and Hela cells were transfected with full-length anti-sense Omi cDNA (+) in pRSC-GFP double expression vector or an empty pRSC-GFP (−) (see FIG. 19). Seventy-two hours after transfection, the cells were treated with Fas (500 ng/ml, 5 hours), TRAIL (1 μg/ml, 5 hours) or staurosporine (1 μM, 5 hours). The percentages of GFP-positive apoptotic cells were determined by fluorescent microscopy after staining with DAPI and propidium iodide. As shown in FIG. 19, the Omi anti-sense cDNA reduced significantly (30-35%) the sensitivity of the transfected cells to apoptotic stimuli. These results indicate that Omi participates together with other apoptotic factors in the overall sensitivity of cells to apoptosis.

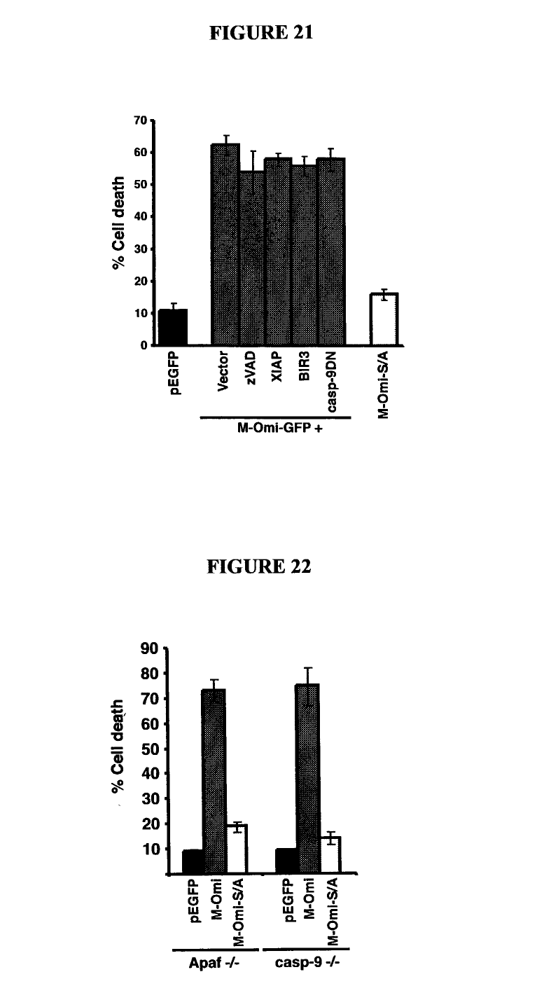

Since Omi possesses both serine protease and IAP-binding activities, the contribution of each of these activities separately towards its overall proapoptotic activity was examined. To determine the contribution of the serine protease activity of Omi towards its proapoptotic activity, it was necessary to express cytosolic protease-active and -inactive forms of Omi which do not bind IAPs in cells. This was achieved by expressing active and inactive (S306A) Omi-GFP fusion proteins without the MTS (M-Omi-GFP and M-Omi-S/A-GFP, respectively) in MCF-7 cells (FIG. 20). It was found that these forms of Omi do not bind XIAP-BIR3 (FIG. 24, first lane) because the initiator methionine before the AVPS motif is not removed in transfected human cells. Nevertheless, only the serine protease active M-Omi-GFP, but not the inactive M-Omi-S/A-GFP, was able to induce apoptosis in MCF-7 cells (FIG. 21). Similar results were obtained with cytosolic Omi variants lacking the AVPS motif.

Further, the Omi apoptotic activity was independent of the cellular caspase activity, as inhibition of cellular caspases with zVAD-FMK, XIAP, XIAP-BIR3 or caspase-9-DN did not block the ability of M-Omi-GFP to induce apoptosis in these cells. Consistent with these results, M-Omi-GFP was also able to induce apoptosis in Apaf −/− and caspase-9 −/− MEFs (FIG. 22). These results indicate that Omi can induce apoptosis in mammalian cells independent of caspases, Apaf-1, or IAPs via its serine protease activity.

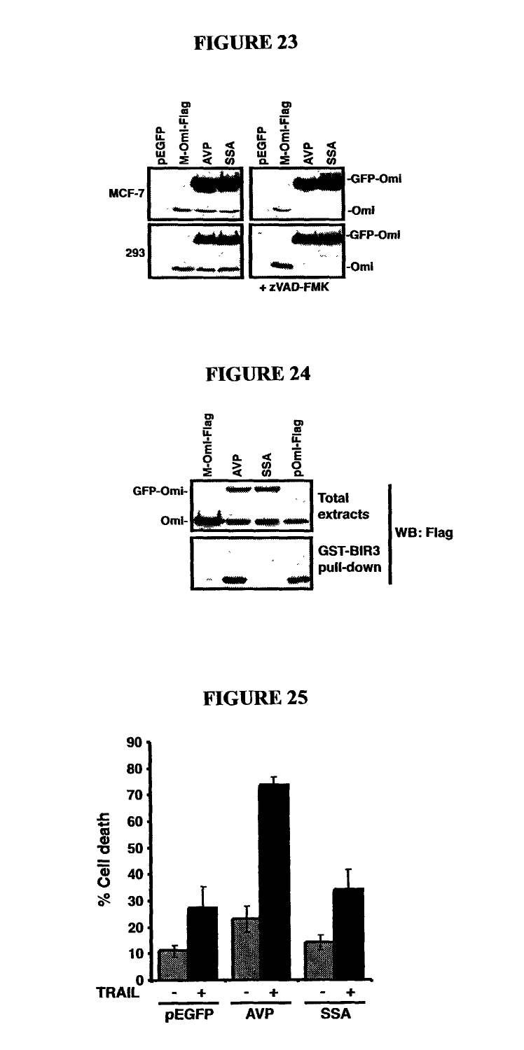

Next, the contribution of the IAP binding activity of Omi to its proapoptotic activity was tested. The MTS of Omi was replaced with a GFP-IETD sequence and mutated at its active site Ser306 to Ala (FIG. 20, GFP-IETD-AVP). Of note, the presence of the IETD sequence between GFP and Omi-S/A allowed caspase-dependent cleavage at the IETD site after the Asp residue and release of mature Omi-S/A in the cytosol of the transfected 293 and MCF-7 cells (FIG. 23). This mature Omi-S/A was able to bind the GST-BIR3 fusion protein (FIG. 24), indicating that it is correctly processed to expose its AVPS motif. Consistent with this result, very little or no processing at the IETD site was observed in the cytosolic extracts of these cells in the presence of the pancaspase inhibitor zVAD-FMK (FIG. 23). As shown in FIG. 25, expression of the GFP-IETD-AVP fusion protein in MCF-7 cells enhanced TRAIL-induced apoptosis in these cells several folds above the GFP transfected control cells. This activity was dependent on the AVPS motif as mutation of the AVPS motif to SSAS motif (GFP-IETD-SSA) abolished the apoptotic enhancement activity of Omi-S/A and the ability to bind to BIR3. These results indicate that the IAP-binding activity of Omi represented by its AVPS motif plays a significant role in its ability to potentiate apoptosis independent of its protease activity. Taken together, Omi not only can induce apoptosis through its serine protease activity but also via its ability to bind and neutralize IAPs.

In providing the forgoing description of the invention, citation has been made to several references that will aid in the understanding or practice thereof. All such references including patents and patent applications are incorporated by reference herein, in their entirety, including those referenced in the Application Data Sheet.

From the foregoing, it will be appreciated that, although specific embodiments of the invention have been described herein for purposes of illustration, various modifications may be made without deviating from the spirit and scope of the invention. In addition, all references including patents, patent applications, and journal articles are incorporated herein in their entirety. Accordingly, the invention is not limited except as by the appended claims.

1. An isolated nucleic acid molecule comprising a polynucleotide having a sequence encoding a peptide or polypeptide of Omi comprising SEQ ID NO:3 and up to 321 contiguous amino acid residues derived from residues 138-458 of SEQ ID NO:1 that specifically binds to a portion of an Inhibitor of Apoptosis Protein (IAP).

2. The isolated nucleic acid molecule of claim 1, wherein said portion comprises a BIR domain.

3. The isolated nucleic acid molecule of claim 2, wherein said BIR domain is BIR1.

4. The isolated nucleic acid molecule of claim 2, wherein said BIR domain is BIR2.

5. The isolated nucleic acid molecule of claim 2, wherein said BIR domain is BIR3.

6. The isolated nucleic acid molecule of claim 1, wherein said binding is to a full-length IAP.

7. An expression vector comprising a nucleic acid molecule of claim 1 operatively linked to regulatory elements.

8. The expression vector of claim 7, wherein the regulatory elements include an inducible or constitutive promoter.

9. A host cell containing an expression vector of claim 7.

10. An isolated peptide or polypeptide comprising SEQ ID NO:3 and up to 321 contiguous amino acid residues derived from residues 138-458 of SEQ ID NO:1 that specifically binds to a portion of an Inhibitor of Apoptosis Protein (IAP).

11. The isolated peptide or polypeptide of claim 10, wherein said portion comprises a BIR domain.

12. The isolated peptide or polypeptide of claim 11, wherein said BIR domain is BIR1.

13. The isolated peptide or polypeptide of claim 11, wherein said BIR domain is BIR2.

14. The isolated peptide or polypeptide of claim 11, wherein said BIR domain is BIR3.

15. The isolated peptide or polypeptide of claim 10, wherein said binding is to a full-length IAP.

16. An isolated nucleic acid molecule comprising a polynucleotide having a sequence encoding a polypeptide of Omi comprising SEQ ID NO:11 and up to 314 additional contiguous amino acid residues derived from residues 138-458 of SEQ ID NO:1, wherein said polypeptide induces caspase-independent apoptosis.

17. An isolated nucleic acid molecule comprising a polynucleotide having a sequence encoding a polypeptide of Omi comprising SEQ ID NO:12, SEQ ID NO:11, and up to 321 contiguous amino acid residues including SEQ ID NO:11 derived from residues 138-458 of SEQ ID NO:1, wherein said polypeptide induces caspase-independent apoptosis.

18. An isolated nucleic acid molecule comprising a polynucleotide having a sequence encoding a polypeptide of Omi comprising SEQ ID NO:3, SEQ ID NO:13, and up to 321 contiguous amino acid residues including SEQ ID NO:13 derived from residues 138-458 of SEQ ID NO:1, wherein said polypeptide fails to have serine protease activity.

19. The isolated nucleic acid molecule of claim 18, wherein Xaa of SEQ ID NO:13 is an alanine.

20. An isolated nucleic acid molecule comprising a polynucleotide having a sequence encoding a polypeptide of Omi comprising the amino acid sequence of residues 134-458 of SEQ ID NO:1 except that the serine at position 306 is mutated and said polypeptide fails to have serine protease activity.

21. The isolated nucleic acid molecule of claim 20, wherein said serine is mutated to an alanine.

22. An isolated polypeptide of Omi comprising SEQ ID NO:11 and up to 314 additional contiguous amino acid residues derived from residues 138-458 of SEQ ID NO:1 wherein said polypeptide induces caspase-independent apoptosis.

23. An isolated polypeptide of Omi comprising SEQ ID NO:12, SEQ ID NO:11, and up to 321 contiguous amino acid residues including SEQ ID NO:11 derived from residues 138-458 of SEQ ID NO:1, wherein said polypeptide induces caspase-independent apoptosis.

24. An isolated polypeptide of Omi comprising SEQ ID NO:3, SEQ ID NO:13, and up to 321 contiguous amino acid residues including SEQ ID NO:13 derived from residues 138-458 of SEQ ID NO:1, wherein said polypeptide fails to have serine protease activity.

25. The isolated polypeptide of claim 24, wherein Xaa of SEQ ID NO:13 is an alanine.

26. An isolated polypeptide of Omi comprising the amino acid sequence of residues 134-458 of SEQ ID NO:1 except that the serine at position 306 is mutated and said polypeptide fails to have serine protease activity.

27. The isolated polypeptide of claim 26, wherein said serine is mutated to an alanine.

28. A method for inducing caspase-dependent apoptosis in a cell, comprising contacting the cell with at least one component selected from the group consisting of:
(a) an Omi peptide or polypeptide of claim 10; and
(b) a nucleic acid molecule of claim 1
under conditions and for a time sufficient to permit the induction of apoptosis in the cell.

29. A method for inducing caspase-independent apoptosis in a cell, comprising contacting the cell with at least one component selected from the group consisting of:
(a) a polypeptide of Omi of claim 22 or 23; and
(b) a nucleic acid molecule of claim 16 or 17
under conditions and for a time sufficient to permit the induction of apoptosis in the cell.

30. A method of identifying an inhibitor or enhancer of caspase-mediated apoptosis comprising:
(a) contacting a cell containing a vector expressing the peptide or polypeptide of claim 10 with a candidate inhibitor or candidate enhancer; and
(b) detecting cell viability,
wherein an increase in cell viability as compared to a control indicates the presence of an inhibitor, and a decrease in cell viability as compared to a control indicates the presence of an enhancer.

31. A method of identifying an inhibitor or enhancer of caspase-mediated apoptosis comprising:
(a) contacting a cell containing a vector expressing the peptide or polypeptide of claim 10 with a candidate inhibitor or candidate enhancer; and
(b) detecting the presence of large and small caspase subunits, and therefrom determining the level of caspase processing activity,
wherein a decrease in processing indicates the presence of an inhibitor, and an increase in processing indicates the presence of an enhancer.

32. The method of claim 31, wherein the caspase detected is selected from the group consisting of caspase-3, caspase-7, and caspase-9.

33. A method of identifying an inhibitor or enhancer of a caspase-mediated apoptosis comprising:
(a) contacting a cell transformed or transfected with a vector expressing the peptide or polypeptide of claim 10 with a candidate inhibitor or candidate enhancer; and
(b) detecting caspase enzymatic activity,
wherein a decrease in enzymatic activity indicates the presence of an inhibitor and an increase in enzymatic activity indicates the presence of an enhancer.

34. The method of claim 33, wherein the caspase enzymatic activity detected is selected from the group consisting of caspase-3, caspase-7 and caspase-9.

35. The method of claim 33, wherein the caspase enzymatic activity is detected by the presence of a substrate cleavage product produced by a caspase cleavage of a substrate.

36. The method of claim 35, wherein said substrate is acetyl DEVD-aminomethyl coumarin.

37. A method of identifying an inhibitor or enhancer of a caspase-independent apoptosis comprising:
(a) contacting a cell transformed or transfected with a vector expressing a polypeptide of claim 22 or 23 with a candidate inhibitor or candidate enhancer; and
(b) detecting cell viability,
wherein an increase in cell viability as compared to a control indicates the presence of an inhibitor, and a decrease in cell viability as compared to a control indicates the presence of an enhancer.

38. A method for identifying a compound that inhibits Omi binding to an Omi-binding molecule, comprising:
(a) contacting a candidate compound with a peptide or polypeptide of claim 10, 22, or 23 in the presence of said Omi-binding molecule; and
(b) detecting displacement or inhibition of binding of said Omi-binding molecule from said Omi peptide or polypeptide.

39. A method for identifying a compound that inhibits Omi binding to a portion of an Inhibitor of Apoptosis Protein (IAP), comprising:
(a) contacting a candidate compound with a peptide or polypeptide of claim 10 in the presence of said portion of an IAP; and
(b) detecting displacement or inhibition of binding of said portion of an IAP from said Omi peptide or polypeptide.

40. The method of claim 39, wherein said portion comprises a BIR domain.

41. The method of claim 40, wherein said BIR domain is BIR1.

42. The method of claim 40, wherein said BIR domain is BIR2.

43. The method of claim 40, wherein said BIR domain is BIR3.

44. The method of claim 39, wherein said contacting is in the presence of a full length IAP.

45. A method for identifying a compound that inhibits Omi binding to a Omi-binding molecule that is not an Inhibitor of Apoptosis Protein (IAP), comprising:
(a) contacting a candidate compound with a peptide or polypeptide of claim 22 or 23 in the presence of said Omi-binding molecule; and
(b) detecting displacement or inhibition of binding of said Omi-binding molecule from said Omi peptide or polypeptide.

46. A method for identifying a compound that inhibits Omi from binding to a Omi-binding molecule, comprising:
(a) contacting a candidate compound with a peptide or polypeptide of claim 10 in the presence of said Omi-binding molecule, at least one caspase or procaspase, cytochrome c, and ATP or dATP; and
(b) performing a functional assay that confirms displacement of said Omi-binding molecule from said Omi peptide or polypeptide.

47. The method of claim 46, wherein the functional assay is carried out in the presence of an initiator caspase or procaspase and an effector procaspase and detects the presence of large and small caspase subunits of said effector procaspase, and therefrom determining the level of caspase processing activity, wherein a decrease in processing confirms displacement.

48. The method of claim 47, wherein said initiator caspase is caspase-9, and the effector procaspase is selected from the group consisting of caspase-3 and caspase-7.

49. The method of claim 47, wherein the functional assay detects the presence of a substrate cleavage product produced by the effector caspase cleavage of a substrate.

50. The method of claim 49, wherein said effector caspase is selected from the group consisting of caspase-3 and caspase-7.

51. The method of claim 49, wherein said substrate is acetyl DEVD-aminomethyl coumarin.

52. A method for identifying a compound that inhibits Omi from binding to a Omi-binding molecule, comprising:
(a) contacting a cell containing a vector expressing the peptide or polypeptide of claim 10 with a candidate compound; and
(b) performing a functional assay that confirms displacement of said Omi-binding molecule from said Omi peptide or polypeptide.

53. The method of claim 52, wherein the functional assay detects the presence of large and small caspase subunits of a procaspase, and therefrom determining the level of caspase processing activity, wherein a decrease in processing confirms displacement.

54. The method of claim 53, wherein the procaspase detected is selected from the group consisting of caspase-3, caspase-7, and caspase-9.

55. The method of claim 52, wherein the functional assay detects the presence of a substrate cleavage product produced by a caspase cleavage of a substrate.

56. The method of claim 55, wherein said substrate is acetyl DEVD-aminomethyl coumarin.

57. An antibody that specifically binds to a peptide or polypeptide of claim 10, 22, or 23.

58. The antibody of claim 57, wherein the antibody is a polyclonal.

59. The antibody of claim 57, wherein the antibody is a monoclonal.

60. An antibody that specifically binds to an epitope located on the N-terminus of Omi.

61. The antibody of claim 60, wherein said epitope comprises SEQ ID NO:3.

62. The antibody of claim 60, wherein said antibody inhibits the binding of Omi to a portion of an IAP.

63. The antibody of claim 62, wherein said portion comprises a BIR domain.

64. The antibody of claim 63, wherein said BIR domain is BIR3.

65. The antibody of claim 62, wherein said antibody inhibits the binding to a full-length IAP.

66. A composition comprising a nucleic acid molecule of claim 1, 16, or 17 and a physiologically acceptable carrier.

67. A composition comprising a peptide of claim 10, 22, or 23 and a physiologically acceptable carrier.

68. A composition comprising an antibody of claim 57 and a physiologically acceptable carrier.

69. A composition comprising an antibody of claim 60 and a physiologically acceptable carrier.

70. An isolated nucleic acid molecule comprising a polynucleotide having a sequence encoding a functional variant of said peptide or polypeptide of claim 1, said variant having at least 50% identity of said peptide or at least 75% identity of said polypeptide up to 75 residues in length or at least 85% identity of said polypeptide over 75 residues in length and said variant specifically binds to a portion of an IAP.

71. An isolated peptide or polypeptide comprising an amino acid sequence comprising a functional variant of said peptide or polypeptide of claim 22, said variant having at least 50% identity of said peptide or at least 75% identity of said polypeptide up to 75 residues in length or at least 85% identity of said polypeptide over 75 residues in length and said variant specifically binds to a portion of an IAP.

72. A method of producing a compound for inhibiting or enhancing caspase-dependent apoptosis in a cell, comprising:
(a) identifying an inhibitor or enhancer of caspase-mediated apoptosis according to a method selected-from the group consisting of claims 30, 31, 33, and 37; and
(b) purifying said inhibitor or enhancer.

73. A method of producing a compound for inhibiting or enhancing apoptosis in a cell, comprising:
(a) identifying a compound that inhibits binding of an Omi peptide or polypeptide according to a method selected from the group consisting of claims 38, 39, 45, 46, and 52; and
(b) purifying said compound.

74. A process for the manufacture of a compound for inhibiting or enhancing caspase-dependent apoptosis in a cell, comprising:
(a) identifying an inhibitor or enhancer of caspase-mediated apoptosis according to a method selected from the group consisting of claims 30, 31, 33, and 37; and
(b) derivitizing the compound of (a) and optionally repeating at least one of steps (a) and (b),
to produce a compound that inhibits or enhances caspase-mediated apoptosis.

75. A process for the manufacture of a compound for inhibiting or enhancing apoptosis in a cell, comprising:
(a) identifying a compound that inhibits binding of an Omi peptide or polypeptide according to a method selected from the group consisting of claims 38, 39, 45, 46, and 52; and
(b) derivitizing the compound of (a) and optionally repeating at least one of steps (a) and (b),
to produce a compound that inhibits binding of an Omi peptide or polypeptide.


SEQUENCE LISTING (17 sequences)

SEQ ID NO: 1 - protein, 458 aa, Homo sapiens
MAAPRAGRGAGWSLRAWRALGGIRWGRRPRLTPDLRALLTSGTSDPRARVTYGTPSLWARLSVGVTEPRACLTSGTPGPRAQLTAVTPDTRTREASENSGTRSRAWLAVALGAGGAVLLLLWGGGRGPPAVLAAVPSPPPASPRSQYNFIADVVEKTAPAVVYIEILDRHPFLGREVPISNGSGFVVAADGLIVTNAHVVADRRRVRVRLLSGDTYEAVVTAVDPVADIATLRIQTKEPLPTLPLGRSADVRQGEFVVAMGSPFALQNTITSGIVSSAQRPARDLGLPQTNVEYIQTDAAIDFGNSGGPLVNLDGEVIGVNTMKVTAGISFAIPSDRLREFLHRGEKKNSSSGISGSQRRYIGVMMLTLSPSILAELQLREPSFPDVQHGVLIHKVILGSPAHRAGLRPGDVILAIGEQMVQNAEDVYEAVRTQSQLAVQIRRGRETLTLYVTPEVTE

SEQ ID NO: 2 - protein, 5 aa, Homo sapiens
AVPIA

SEQ ID NO: 3 - protein, 4 aa, Homo sapiens
AVPS

SEQ ID NO: 4 - protein, 15 aa, Drosophila sp.
AVAFYIPDQATLLRE

SEQ ID NO: 5 - protein, 15 aa, Drosophila sp.
AIAYFIPDQAQLLAR

SEQ ID NO: 6 - protein, 15 aa, Drosophila sp.
AVPFYLPEGGADDVA

SEQ ID NO: 7 - protein, 15 aa, Homo sapiens
ATPFQEGLRTFDQLD

SEQ ID NO: 8 - protein, 15 aa, Homo sapiens
AVPIAQKSEPHSLSN

SEQ ID NO: 9 - protein, 15 aa, Homo sapiens
AVPSPPPASPRSQYN

SEQ ID NO: 10 - protein, 4 aa, Artificial Sequence
  Amino acid residues that replace the AVPS motif in a non-IAP binding Omi mutant
VVAS

SEQ ID NO: 11 - protein, 7 aa, Homo sapiens
GNSGGPL

SEQ ID NO: 12 - protein, 5 aa, Artificial Sequence
  Fusion of Met to Omi N-terminus
MAVPS

SEQ ID NO: 13 - protein, 7 aa, Artificial Sequence
  Mutant of Omi serine protease domain
GNXGGPL

SEQ ID NO: 14 - DNA, 1374 bp, Homo sapiens
atggctgcgccgagggcggggcggggtgcaggctggagccttcgggcatggcgggctttggggggcattcgctgggggaggagaccccgtttgacccctgacctccgggccctgctgacgtcaggaacttctgacccccgggcccgagtgacttatgggacccccagtctctgggcccggttgtctgttggggtcactgaaccccgagcatgcctgacgtctgggaccccgggtccccgggcacaactgactgcggtgaccccagataccaggacccgggaggcctcagagaactctggaacccgttcgcgcgcgtggctggcggtggcgctgggcgctgggggggcagtgctgttgttgttgtggggcgggggtcggggtcctccggccgtcctcgccgccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgcccatgtggtggctgatcggcgcagagtccgtgtgagactgctaagcggcgacacgtatgaggccgtggtcacagctgtggatcccgtggcagacatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactctggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccagcggcgctacattggggtgatgatgctgaccctgagtcccagcatccttgctgaactacagcttcgagaaccaagctttcccgatgttcagcatggtgtactcatccataaagtcatcctgggctcccctgcacaccgggctggtctgcggcctggtgatgtgattttggccattggggagcagatggtacaaaatgctgaagatgtttatgaagctgttcgaacccaatcccagttggcagtgcagatccggcggggacgagaaacactgaccttatatgtgacccctgaggtcacagaa

SEQ ID NO: 15 - protein, 361 aa, Homo sapiens
MAAPRAGRGAGWSLRAWRALGGIRWGRRPRLTPDLRALLTSGTSDPRARVTYGTPSLWARLSVGVTEPRACLTSGTPGPRAQLTAVTPDTRTREASENSGTRSRAWLAVALGAGGAVLLLLWGGGRGPPAVLAAVPSPPPASPRSQYNFIADVVEKTAPAVVYIEILDRHPFLGREVPISNGSGFVVAADGLIVTNAHVVADRRRVRVRLLSGDTYEAVVTAVDPVADIATLRIQTKFGNSGGPLVNLDGEVIGVNTMKVTAGISFAIPSDRLREFLHRGEKKNSSSGISGSQRRYIGVMMLTLSPRAGLRPGDVILAIGEQMVQNAEDVYEAVRTQSQLAVQIRRGRETLTLYVTPEVTE

SEQ ID NO: 16 - DNA, 641 bp, Homo sapiens
  misc_feature 530: n = A,T,C or G
gttgctaggtataattttattttatataaaaagtgtttctgtgattcttcagagcccaggagtcagtgctggtggttggagggacctgcccccactggttcatttaaccctctgtctcggtgccctcagaacctcagccagaaaggcaaggaggaaatcagagcaggagcctcatactcttggtgatctattcattctgtgacctcaggggtcacatataagggtcagtgtttctcgtccccgccggatctgcactgccaactgggattgggttcgaacagcttcataaacatcttcagcattttgtaccatctgctccccaatggccaaaatcacatcaccaggccgcagaccagcccggtgtgcaggggagcccaggatgactttatggatgagtacaccatgctgaacatcgggaaagctggttctcgaagctgagttcagcaagggatgctggactcagggtcagcatcatcaccccaatgtagcgccgctgggacccactgattccggaggaggattcttctttncccacgatgcagaaactcttcgaggacatcagaaaggatggccaaggagattccagctgtgacctttcatgtgttacttccatcaacctccccattcaggttaaccagggg

SEQ ID NO: 17 - protein, 5 aa, Homo sapiens
  VARIANT (4)...(4): Xaa = Arg, Gln or Gly
QACXG